(19) United States
(12) Patent Application Publication
(10) Pub. No.: US 2021/0112816 A1
(43) Pub. Date: Apr. 22, 2021

(54) PLASMA IMMOBILIZATION OF BACTERIOPHAGES AND APPLICATIONS THEREOF

(71) Applicant: PHAGELUX (CANADA) INC., Montreal (CA)

(72) Inventor: Nancy TAWIL, Cantley (CA)

(21) Appl. No.: 16/606,585

(22) PCT Filed: Apr. 25, 2018
(86) PCT No.: PCT/IB2018/052880

§ 371 Date: Oct. 18, 2019

Related U.S. Application Data
(60) Provisional application No. 62/490,291, filed on Apr. 26, 2017.

(51) Int. Cl. A01N 63/40; A61L 27/06; A61L 27/34; A61L 27/54; A61L 29/10; A61L 29/08; A61L 29/16; A61L 31/02; A61L 31/10; A61L 31/16

(52) U.S. Cl. CPC A01N 63/40; A61L 27/06; A61L 27/34; A61L 27/54; A61L 29/106; A61L 2300/404; A61L 29/16; A61L 31/022; A61L 31/10; A61L 31/16; A61L 2300/258; A61L 29/085

(57) ABSTRACT

A medical device, the medical device including a substrate defining a surface; a plasma polymer layer bound to and coating the surface; and a bactericide layer bound to the plasma polymer layer, the plasma polymer layer being between the substrate and the bactericide layer. Also, a method for coating a surface of a substrate of a medical device with a bactericide layer, the method including: exposing the surface to a plasma to form a plasma polymer layer bound to the surface; and binding a bactericide layer to the plasma polymer layer.

### FIELD OF THE INVENTION

The present invention relates to the general field of bacteriophages and is more particularly concerned with plasma immobilization of bacteriophages and applications thereof.

### BACKGROUND

Bacteriophages are viruses that specifically infect bacteria. They are the most predominant biocontrol agents in the environment, and their capacity to infect only a limited number of bacterial hosts can be exploited in numerous medical, industrial, and ecological applications.

The global, ever-increasing occurrence of multidrug-resistant infections is alarming with attributable health care costs $20 billion in the United States alone. Although substantial efforts are directed toward producing antibiotic and non-antibiotic derivatives, such as antibacterial vaccines, immunostimulants, adjuvants, and probiotics; few drugs are under development. Moreover, the reintroduction of silver products as treatment alternatives for wounds has brought about the development of bacterial resistance genes to silver. These factors have prompted a renewed interest in phage therapy worldwide with early stage clinical studies confirming the safety of bacteriophage use.

Microbiological and resistance epidemiology of periprosthetic joint infections show that the vast majority of organisms present are methicillin-resistant and methicillin-sensitive S. aureus, and methicillin-resistant and methicillin-sensitive Staph. epidermidis. In fact, 3 out of 4 implant-related infections are caused by Staphylococcal species, with S. aureus being the main causative agent in orthopedics. Infections caused by all existing pathogenic microbial species except staphylococci represent together only a minority of implant infections, just about 22%. The Staphylococcus genus therefore acquires a huge importance in implant-related infections.

Antibiotic resistance is currently a main issue requiring primary clinical attention. Many important pathogens, S. aureus in first line among them, have long been recognized to exhibit always more alarming levels of antibiotic resistance. Moreover, bacteria forming biofilms on prosthetic surfaces are particularly resistant to antimicrobials and tend to survive to aggressive chemotherapy even in the absence of specific antibiotic resistance factors. In consideration of this, there is a need to find alternative means of effectively treating implant associated infections.

Accordingly, there exists a need for a way to give antibacterial properties to biomaterials.

### SUMMARY OF THE INVENTION

Periprostethic infections impose a significant burden on the patient and the health care system. Two-stage revision with interval placement of an antibiotic-cement spacer is the standard of care for chronic periprosthetic infections. We propose using this technology in spacer implants as a means to directly deliver bacteriophages to the infected tissues for a prolonged period of time, and maintains soft tissue tension to facilitate the re-implantation procedure by ways of a phage-coated spacer implant.

The past decade has also seen a shift from biostable to biodegradable implants and dressings. Notably, the field of tissue engineering has focused on the development of biological substitutes that not only maintain, but restore and improve tissue function. Moreover, antibacterial properties are desirable for most biomaterials as they are easily colonized by biofilm-forming bacteria.

Design of implants demands an interface that is active towards bacteriophages, and that can provide favorable physical and chemical environment in order to regulate the behavior and biological functions of microorganisms. In this regard, modifying surfaces with plasma is considered as one of the most efficient methods through which we can trigger and enhance surface immobilization through functional group grafting or deposition of a new functional layer.

The present invention proposes a method of treating a substrate with a plasma and subsequent immobilization of phages or phage-derived proteins on the surface.

In some embodiments, the substrate is made of a suitable polymer, such as a Polyester amide urea (PEAU), a leucine-based poly ester amide polymer, or another amino acid based copolymer. Due to both groups, ester and amide, such polymers are biodegradable (ester group) and have good thermal stability and mechanical strength (amide group with strong intermolecular interactions). The incorporation of leucine, or other suitable amino acid, improves the biocompatibility of the polymer.

Such a polymer is synthesized, in some embodiments, by interfacial polycondensation of the monomer L6, di-p-sulfonic acid salt of bis-(L-leucine)-1,6-hexylene diester with trisphogene/sebacoyl chloride with water/dichloromethane system. The use of dichloromethane allows direct utilization of the biocomposite for phages incorporation therefore for microspheres fabrication. This method is fast, irreversible, involves two immiscible phases at room temperature and lead to high molecular weight polymer. Synthesis of the monomer L6 was executed in the presence of p-toluene sulfonic acid by condensation of L-leucine with 1,6-hexanediol in refluxed cyclohexane, because it is less toxic than solvents such as benzene. Purification includes recrystallization from water, filtration and drying under vacuum.

Other polymers usable as substrate in the invention include:

A polymer selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond,

further wherein
the at least one diol is a compound of formula:

HO—R₁—OH, R₁ is chosen from C₂-C₁₂ alkylene optionally interrupted by at least one oxygen, C₃-C₈ cycloalkylene, C₃-C₁₀ cycloalkylalkylene,

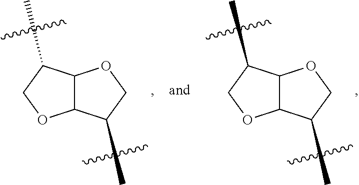

the at least one diacid is a compound of formula:

HO—(CO)—R₃—(CO)—OH, R₃ is C₂-C₁₂ alkylene,
the at least one amino acid is chosen from naturally occurring amino acids and non-naturally occurring amino acid.

In some embodiments, the polymer is selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond, and
(4) a poly (ester amide urethane) wherein at least one diol, at least one diacid, and at least one amino acid are linked together through an ester bond, an amide bond, and a urethane bond,
wherein the at least one diol, at least one diacid, and at least one amino acid are as defined above.

In some more specific embodiments of the invention, the polymer is a poly (ester amide urea) comprising the following two blocks with random distribution thereof:

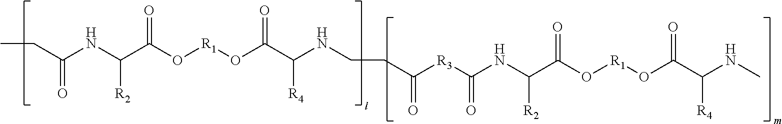

wherein
the ratio of l:m ranges from 0.05:0.95 to 0.95:0.05, l+m=1, R₁ is chosen from C₂-C₁₂ alkylenes optionally interrupted by at least one oxygen, C₃-C₈ cycloalkylenes, C₃-C₁₀ cycloalkylalkylenes,

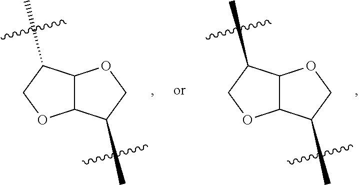

R₃ is C₂-C₁₂ alkylene,
R₂ and R₄ are independently chosen from the side chains of L- and D-amino acids so that the carbon to which R₂ or R₄ is attached has L or D chirality.

In some more specific embodiments of the invention, the polymer is poly (ester urethane urea) comprising the following two blocks with random distribution thereof:

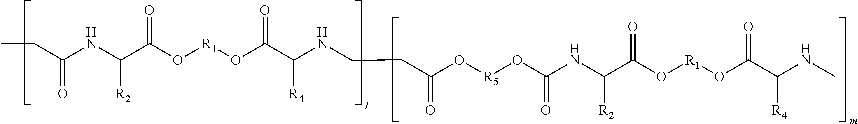

wherein

the ratio of l:m ranges from 0.05:0.95 to 0.95:0.05, l+m=1, R₁ and R₅ are independently chosen from C₂-C₁₂ alkylenes optionally interrupted by at least one oxygen, C₃-C₈ cycloalkylenes, C₃-C₁₀ cycloalkylalkylenes,

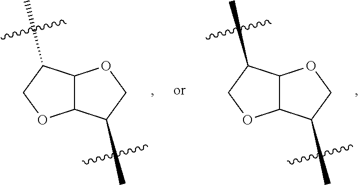

and
R₂ and R₄ are independently chosen from the side chains of L- and D-amino acids so that the carbon to which R₂ or R₄ is attached has L or D chirality.

In some more specific embodiments of the invention, the polymer is poly (ester amide urethane urea) comprising the following three blocks with random distribution thereof:

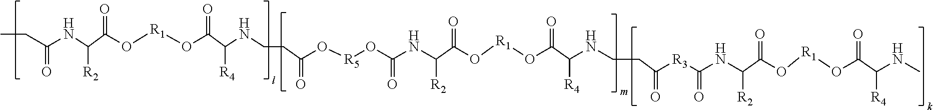

wherein

the ratio of l:m:k ranges from 0.05:0.05:0.90 to 0.90:0.05:0.05, l+m+k=1, R₁ and R₅ are independently chosen from C₂-C₁₂ alkylenes optionally interrupted by at least one oxygen, C₃-C₈ cycloalkylenes, C₃-C₁₀ cycloalkylalkylenes,

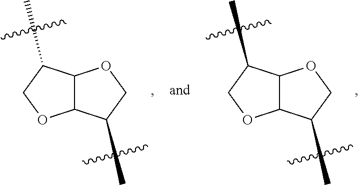

R₃ is C₂-C₁₂ alkylene, and
R₂ and R₄ are independently chosen from the side chains of L- and D-amino acids so that the carbon to which R₂ or R₄ is attached has L or D chirality.

In some more specific embodiments of the invention, the polymer is (ester amide urethane) comprising the following two blocks with random distribution thereof:

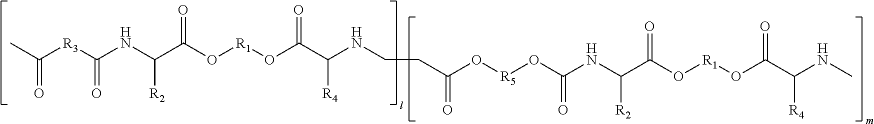

wherein
the ratio of l:m ranges from 0.05:0.95 to 0.95:0.05, l+m=1,
R₁ and R₅ are independently chosen from C₂-C₁₂ alkylenes optionally interrupted by at least one oxygen, C₃-C₈ cycloalkylene, C₃-C₁₀ cycloalkylalkylene,

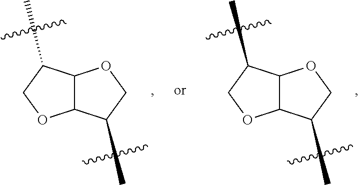

R₃ is C₂-C₁₂ alkylene, and
R₂ and R₄ are the same and selected from the side chains of L- and D-amino acids so that the carbon to which R₂ or R₄ is attached has L or D chirality.

In the above polymers, in some very specific embodiments of the invention, one or more of the following hold: R₁ is —(CH₂)₆—, R₃ is —(CH₂)₈—, or both R₂ and R₄ are the side chain of L-leucine.

Blends of the above-mentioned polymers are also usable in the preparation of the substrate of the present invention.

More details regarding such polymers and others usable as substrate in the present invention are provided in PCT application PCT/IB2016/001006 filed Jun. 21, 2016, the contents of which is hereby incorporated by reference in its entirety.

In a broad aspect, there is provided a method for immobilizing bacteriophages on a substrate, the method comprising: treating the substrate with a plasma to form a treated substrate; and coating the treated substrate with phages. There may also be provided a method wherein the plasma is a cold plasma.

There may also be provided a method wherein the plasma includes nitrogen.

There may also be provided a method wherein the plasma includes at least one of N₂ and NH₃.

There may also be provided a method wherein the plasma includes N₂ and H₂.

There may also be provided a method wherein treating the surface includes forming reactive groups on the surface of the substrate, the reactive groups being selected from the group consisting of primary amines, secondary amines, tertiary amines, amides and combinations thereof.

There may also be provided a method wherein the plasma includes oxygen.

There may also be provided a method wherein treating the surface includes forming reactive groups on the surface of the substrate, the reactive groups being selected from the group consisting of carboxylic groups, hydroxyls, ketones, aldehydes, and esters.

There may also be provided a method wherein the plasma includes at least one of CO and CO₂.

There may also be provided a method wherein treating the surface includes forming reactive groups on the surface of the substrate, the reactive groups being selected from the group consisting of COOH, peroxide and OH.

The invention may also provide a method wherein the plasma includes Ar.

There may also be provided a method wherein treating the surface includes forming free radicals on the surface of the substrate.

There may also be provided a method wherein the plasma includes at least one of Ar, He, O₂, N₂, NH₃, and CF₄.

There may also be provided a method wherein treating the surface includes forming free radicals on the surface of the substrate.

There may also be provided a method wherein the plasma includes at least one of Ar and He, treating the surface includes forming free radicals on the surface of the substrate, the method further comprising exposing the free radicals to a gas including oxygen to initiate a polymerization reaction.

There may also be provided a method wherein the substrate is selected from the group consisting of a substrate including biodegradable amino-acid based polymers, a commercial gauze, a biological material and a metallic implant.

There may also be provided a method wherein the plasma includes at least one of NH₃, N₂/H₂, He, O₂, Ar, N₂, O₂, CO, CO₂, NO, NO₂, SO₂, Ne, H₂ and air.

In another broad aspect, there is provided a substrate treated with a plasma as recited above and a substrate treated with a plasma as recited above with phages immobilized thereon.

In a broad aspect, there is provided a method for immobilizing bacteriophages on a substrate, the method comprising: treating the substrate with a plasma to form a treated substrate; and coating the treated substrate with phages. The surface may include titanium. There may also be provided a method wherein the plasma is a carboxyl plasma or a nitrogen plasma, but other types of plasma are within the scope of the invention.

In another broad aspect, there is provided a substrate treated with a plasma as recited with phages immobilized thereon.

The invention may also provide a method wherein the bacteriophages are covalently immobilized on a thin plasma polymer layer with tunable properties.

In another broad aspect, there is provided a strategy consisting of dip-coating the implant in a block copolymer containing bacteriophages following plasma surface activation. This coating is shown to possess many micro-channels that allow easy transport of bacteriophages to the top (outer) surface, where some portion of the phages is required immediately. These micro-channels can be created by leaching of incorporated salt crystals, among other possible approaches.

Another strategy consists of spraying the substrate with one of two different formulations containing microencapsulated bacteriophages. One the principal virtues of this method will be to protect the underlying bacteriophages and microspheres against possible removal or other damage during handling, packaging, implantation by the surgeon, etc.

In another broad aspect, there is provided a medical device, the medical device comprising: a substrate defining a surface; a plasma polymer layer bound to and coating the surface; and a bactericide layer bound to the plasma polymer layer, the plasma polymer layer being between the substrate and the bactericide layer.

There may also be provided a medical device wherein the bactericide layer includes bioactive bacteriophages.

There may also be provided a medical device wherein the bactericide layer includes bacteriophage related products selected from the group consisting of endolysins, lysostaphins, phage proteins, phage enzymatic formulations, and combinations thereof.

There may also be provided a medical device wherein the bactericide layer further includes a bioactive agent selected from the group consisting of antibiotics, a cell adhesion promoting agents, an antithrombic factors, antiseptics, anti-infectives, antibiotics, pain relievers, antibacterials, antiprotozoal agents, antiviral agents, analgesics, anti-inflammatory agents, contraceptives, CNS active drugs, hormones, enzymes, hemostatics, and vaccines.

There may also be provided a medical device wherein the substrate includes a metal or a metal alloy.

There may also be provided a medical device wherein the substrate includes titanium.

There may also be provided a medical device wherein the substrate is essentially made of titanium.

There may also be provided a medical device wherein the substrate includes at least one of a polymer, iron, copper, zinc, lead, aluminum, titanium, gold, platinum, silver, cobalt, chromium, vanadium, tantalum, nickel, magnesium, manganese, cobalt chrome, nickel titanium, titanium vanadium aluminum, and stainless steel.

There may also be provided a medical device wherein the plasma polymer layer is between 10 and 1000 nm thick.

There may also be provided a medical device wherein the plasma polymer layer is between 100 and 500 nm thick.

There may also be provided a medical device wherein the bactericide layer includes bioactive bacteriophages covalently bound to the plasma polymer layer, optionally a combination of both podoviridae and myoviridae in the same bactericide layer.

There may also be provided a medical device wherein the bactericide layer includes bacteriophage related products covalently bound to the plasma polymer layer.

There may also be provided a medical device wherein the bactericide layer includes a coating material covalently bound to the plasma polymer layer.

There may also be provided a medical device wherein the bactericide layer includes a coating material electrostatically bound to the plasma polymer layer.

There may also be provided a medical device wherein the bactericide layer includes bioactive bacteriophages dispersed in a coating material, the coating material being bound to the plasma polymer.

There may also be provided a medical device wherein the bactericide layer includes bacteriophage related products dispersed in a coating material, the coating material being bound to the plasma polymer.

There may also be provided a medical device wherein the coating material is a polymer defining an exposed surface, the bactericide layer defining microchannels extending in the coating material from the exposed surface.

There may also be provided a medical device wherein the microchannels have a diameter of from about 5 nm to about 5 μm.

There may also be provided a medical device further comprising salt crystals embedded in the coating material.

There may also be provided a medical device wherein bacteriophages are adsorbed on the salt crystals.

There may also be provided a medical device wherein the salt crystals include at least one of calcium salt crystals, magnesium salt crystals, strontium salt crystals, and barium salt crystals.

There may also be provided a medical device wherein the salt crystals are between about 5 nm and about 5 μm in size.

There may also be provided a medical device wherein the coating material is a block co-polymer.

There may also be provided a medical device wherein the bactericide layer includes bacteriophage-containing biodegradable microcapsules bound to the plasma polymer.

There may also be provided a medical device wherein the bactericide layer includes bacteriophage-containing biodegradable microcapsules embedded in a coating material, the coating material being bound to the plasma polymer, the bactericide layer optionally including bacteriophages dispersed thereinto outside of the microcapsules.

There may also be provided a medical device wherein the biodegradable microcapsules are made of a co-polymer.

There may also be provided a medical device wherein the coating material includes Poloxamer 407.

There may also be provided a medical device wherein the coating material includes polyvinyl alcohol (PVA).

There may also be provided a medical device wherein the medical device is selected from the group consisting of an orthopaedic implant, a stent, a catheter, and a defibrillator.

In yet another broad aspect, there is provided a method for coating a surface of a substrate of a medical device with a bactericide layer, the method comprising: exposing the surface to a plasma to form a plasma polymer layer bound to the surface; and binding a bactericide layer to the plasma polymer layer.

There may also be provided a method wherein the bactericide layer includes bioactive bacteriophages.

There may also be provided a method wherein the bactericide layer includes bacteriophage related products selected from the group consisting of endolysins, lysostaphins, phage proteins, phage enzymatic formulations, and combinations thereof.

There may also be provided a method wherein the plasma is a cold plasma.

There may also be provided a method wherein the plasma includes nitrogen.

There may also be provided a method wherein the plasma includes at least one of N₂ and NH₃.

There may also be provided a method wherein the plasma includes N₂ and H₂.

There may also be provided a method wherein exposing the surface to the plasma to form the plasma polymer layer includes forming reactive groups on the surface of the substrate, the reactive groups being selected from the group consisting of primary amines, secondary amines, tertiary amines, amides and combinations thereof.

There may also be provided a method wherein the plasma includes oxygen.

There may also be provided a method wherein exposing the surface to the plasma to form the plasma polymer layer includes forming reactive groups on the surface of the substrate, the reactive groups being selected from the group consisting of carboxylic groups, hydroxyls, ketones, aldehydes, and esters.

There may also be provided a method wherein the plasma includes at least one of CO and CO₂.

There may also be provided a method wherein exposing the surface to the plasma to form the plasma polymer layer includes forming reactive groups on the surface of the substrate, the reactive groups being selected from the group consisting of COOH, peroxide and OH.

There may also be provided a method wherein the plasma includes Ar.

There may also be provided a method wherein exposing the surface to the plasma to form the plasma polymer layer includes forming free radicals on the surface of the substrate.

There may also be provided a method wherein the plasma includes at least one of NH₃, N₂/H₂, He, O₂, Ar, N₂, O₂, CO, CO₂, NO, NO₂, SO₂, Ne, H₂, air and CF₄.

There may also be provided a method wherein exposing the surface to the plasma to form the plasma polymer layer includes forming free radicals on the surface of the substrate.

There may also be provided a method wherein the plasma includes at least one of Ar and He, and wherein exposing the surface to the plasma to form the plasma polymer layer includes forming free radicals on the surface of the substrate, the method further comprising exposing the free radicals to a gas including oxygen to initiate a polymerization reaction.

There may also be provided a method wherein the substrate is selected from the group consisting of a substrate including polymers, biodegradable amino-acid based polymers, a commercial gauze, a metal, and an alloy.

There may also be provided a method wherein the plasma includes at least one of Acetic acid, 4-vinylpyridine, 1-vinylimidazole, an acrylate, ethyl lactate, ethylene, lactic acid, e-caprolactone, methanol, water, allylamine, ethylenediamine, acrylic acid, hydroxymethylmetacrylate, propylethylglycol, hexamethyldisyloxane, aminosilanes, carboxylsilanes, hydroxylsilanes and mercaptosilanes.

There may also be provided a method wherein the plasma is an atmospheric pressure plasma.

There may also be provided a method wherein the plasma is a low pressure plasma.

There may also be provided a method wherein exposing the surface to the plasma to form the plasma polymer layer includes growing the plasma polymer layer until the plasma polymer layer is between 10 nm and 1000 nm thick.

There may also be provided a method wherein exposing the surface to the plasma to form the plasma polymer layer includes growing the plasma polymer layer until the plasma polymer layer is between 100 nm and 500 nm thick.

There may also be provided a method wherein binding the bactericide layer to the plasma polymer layer includes covalently binding bioactive bacteriophages to the plasma polymer layer.

There may also be provided a method wherein binding the bactericide layer to the plasma polymer layer includes contacting the substrate coated with the plasma polymer layer with a suspension including bioactive bacteriophages.

There may also be provided a method wherein binding the bactericide layer to the plasma polymer layer includes dip-coating the substrate coated with the plasma polymer layer with in suspension including bioactive bacteriophages.

There may also be provided a method wherein binding the bactericide layer to the plasma polymer layer includes solvent casting a suspension including bioactive bacteriophages on the plasma polymer layer.

There may also be provided a method wherein binding the bactericide layer to the plasma polymer layer includes covalently binding bacteriophage related products to the plasma polymer layer.

There may also be provided a method wherein binding the bactericide layer to the plasma polymer layer includes contacting the substrate coated with the plasma polymer layer in a suspension or a solution including bacteriophage related products.

There may also be provided a method wherein binding the bactericide layer to the plasma polymer layer includes binding a coating material in which bioactive bacteriophages are dispersed to the plasma polymer layer.

There may also be provided a method wherein binding the bactericide layer to the plasma polymer layer includes binding a coating material in which bacteriophage related products are dispersed to the plasma polymer layer.

There may also be provided a method wherein binding the bactericide layer to the plasma polymer layer includes contacting a solution including the coating material in a solvent with the plasma polymer layer and subsequently evaporating the solvent.

There may also be provided a method wherein the coating material is a polymer defining an exposed surface, the method further comprising forming microchannels extending in the coating material from the exposed surface.

There may also be provided a method wherein forming the microchannels includes etching the exposed surface with a plasma.

There may also be provided a method wherein salt crystals are dispersed in the coating material, forming the microchannels includes leaching the salt crystals from the coating material.

There may also be provided a method wherein leaching the salt crystals is performed while contacting the coating material with biological tissues when the medical device is in use in a subject.

There may also be provided a method wherein the coating material is a block co-polymer.

There may also be provided a method wherein binding the bactericide layer to the plasma polymer layer includes binding to the plasma polymer layer a coating material in which bacteriophage-containing biodegradable microcapsules are embedded.

There may also be provided a method wherein the biodegradable microcapsules are made of a co-polymer.

There may also be provided a method wherein the coating material includes Poloxamer 407.

There may also be provided a method wherein the coating material includes polyvinyl alcohol (PVA).

There may also be provided a method wherein binding the bactericide layer to the plasma polymer layer includes spraying a coating material including a suspension of microcapsules in which bioactive bacteriophages are dispersed on the plasma polymer layer.

The method as defined in any one of claims 30 to 71, wherein the medical device is selected from the group consisting of an orthopaedic implant, a stent, a catheter, and a defibrillator.

The present document refers to a number of documents, the contents of which is hereby incorporated by reference in its entirety. The present patent application claims priority from U.S. Provisional Patent Application 62/490,291 filed Apr. 26, 2017, the contents of which is hereby incorporated by reference in its entirety.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the drawings:

FIG. 1, in a photograph, illustrates petri dishes including samples treated in accordance with the invention and controls after incubation for PEAU TMN samples tested on SaX (left) and SaA12 (right). 3: treated with plasma LP COOH gas, 4: treated with plasma LP COOH gas+phages, CTRL: non-treated;

FIG. 2, in a photograph, illustrates petri dishes including samples treated in accordance with the invention and controls after incubation for PEAU TMN samples tested on SaX (left) and SaA12 (right). 3: treated with plasma LP NH₃, 4: treated with plasma LP NH₃+phages, CTRL: non-treated;

FIG. 3, in a photograph, illustrates petri dishes including samples treated in accordance with the invention and controls after incubation for PEAU TMN samples tested on SaX (left) and SaA12 (right). 3: treated with plasma AP room air, 4: treated with plasma AP room air+phages, CTRL: non-treated;

FIG. 4, in a photograph, illustrates petri dishes including samples treated in accordance with the invention and controls after incubation for Formamedic gauze samples tested on SaX (left) and SaA12 (right). 11: treated with plasma LP NH₃, 12: treated with plasma LP NH₃+phages, CTRL: non-treated;

FIG. 5A, in a schematic representation, illustrates a medical device in accordance with an embodiment of the present invention;

FIG. 5B, in a schematic representation, illustrates a medical device in accordance with an other embodiment of the present invention;

FIG. 5C, in a schematic representation, illustrates a medical device in accordance with yet an other embodiment of the present invention;

FIG. 5D, in a schematic representation, illustrates a medical device in accordance with yet an other embodiment of the present invention;

FIG. 5E, in a schematic representation, illustrates a medical device in accordance with yet an other embodiment of the present invention;

FIG. 5F, in a schematic representation, illustrates a medical device in accordance with yet an other embodiment of the present invention;

FIG. 6: Physico-chemical analysis of miniature knee implant treated with S. aureus bacteriophage. a) Miniature knee implant. b) FEG-SEM image of implant. c) AFM adhesion micrograph, arrows point to covalently immobilized bacteriophages. d) AFM height sensor 3D reconstruction, arrows point to covalently immobilized bacteriophages.

FIG. 7: AFM micrographs of titanium rods. a) Height sensor AFM 3D reconstruction of control (no treatment) titanium rod. b) Adhesion photomicrograph of control rod. c) Height sensor AFM 3D reconstruction of rod with covalently immobilized bacteriophages. d) AFM adhesion photomicrographs showing covalently immobilized bacteriophages.

FIG. 8: Bacterial lawn assay. Control petri dishes for “No Treatment”, “Plasma Treated”, “Plasma treated+TMN buffer”, and “Plasma treated+PBS buffer” show no lysis on the S. aureus bacterial lawn. Treatment groups “Plasma treated+phages BP39” and “Plasma treated+Staph endolysin” group show lysis of S. aureus bacterial lawn on the periphery of the treated implant.

FIG. 9: Attachment assay. Black (left): Control (no treatment); Red (middle): Plasma control (no phages); Blue (right): Plasma immobilized phages on titanium rods.

FIG. 10: Liquid proliferation assay. Black (left): Control (no treatment); Red (middle): Plasma control (no phages); Blue (right): Plasma immobilized phages on titanium rods.

FIG. 11: Bacterial lawn assay. Upper two petri dishes are control groups for PPE:N and PPE:O plasma treatments, no phages were applied. Bottom two petri dishes: Titanium rods where either treated with PPE:N (left) or PPE:O (right) following subsequent immobilization of bacteriophages. Clear lysis of the bacterial lawn appears around the treated titanium rods.

FIG. 12: Liquid proliferation assay. Blue (two left bars): PPE:N plasma treated groups. Red (two right bars) PPE:O plasma treated groups. Subsequent phage immobilization result in 2.76 and 2.40 log reduction of bacterial proliferation respectively.

FIG. 13: Soft agar proliferation assay. Black (first bar): non-treated sample. Blue (second and third bars): PPE:N plasma treated groups. Subsequent phage immobilization result in 1.87 log reduction of bacterial proliferation.

FIG. 14: Attachment assay for PPE:N and J21-P1 Left petri: control group titanium rod with no plasma treatment. Middle petri dish show control titanium rod treated with PPE:N plasma and TMN buffer. Right petri dish show titanium rod treated with PPE:N plasma and bacteriophage J21-P1. Clear lysis of the bacterial lawn appears around the phage treated titanium rods.

FIG. 15: Atomic force micrographs for untreated Ti-rods (left) and rods treated with plasma following immobilization of phages (right). Arrows point to immobilized phages.

FIG. 16: Coating stability at 2 weeks post-treatment. Left petri dish control group titanium rod with treated with PPE:N plasma and TMN buffer. Right petri dish show titanium rod treated with PPE:N plasma and bacteriophage J21-P1. Clear lysis of the bacterial lawn appears around the phage treated titanium rods 2 weeks post-treatment.

FIG. 17: Solid proliferation assay 2 weeks post treatment. Black: non-treated sample. Red: PPE:N plasma+TMN buffer control group. Blue: PPE:N plasma+phage treated groups. Subsequent phage immobilization result in 0.74 log reduction of bacterial proliferation2 weeks post-treatment.

FIG. 18: Biofilm optical density following treatment of titanium coupons. (1) Ctl− group is the negative control (2) Ctrl+group was treated with plasma+TMN buffer; (3) F11/R=2 was treated with plasma R=2 and F11 microencapsulated spray formulation; (4) Ckt#3/R=2 group was treated with plasma treatment R=2 and phage cocktail; (5) F11/R=2 was treated with plasma R=8 and F11 microencapsulated spray formulation; 4) Ckt#3/R=8 group was treated with plasma treatment R=8 and phage cocktail; and

FIG. 19: Biofilm optical density following treatment of 316L stainless steel coupons. (1) Ctl− group is the negative control (2) Ctrl+group was treated with plasma+TMN buffer; (3) F11/R=2 was treated with plasma R=2 and F11 microencapsulated spray formulation; (4) Ckt#3/R=2 group was treated with plasma treatment R=2 and phage cocktail; (5) F11/R=2 was treated with plasma R=8 and F11 microencapsulated spray formulation; 4) Ckt#3/R=8 group was treated with plasma treatment R=8 and phage cocktail.

### DETAILED DESCRIPTION

Surface immobilization of bacteriophages and phage product is useful in some biomedical applications. In particular, initial physico-chemical surface modification may significantly improve the substrate-phage interaction, while maintaining the phage's viability and ability to infect and kill its bacterial host. In some cases, it is advantageous to avoid the use of hazardous organic solvents, which might inactivate phages and impose problems toward cell viability, or to at least minimize exposition to such solvents. Moreover, it is desirable in some applications to maintain a good control over the modified surface in terms of thickness and chemical composition.

Plasma is a partly ionized gas containing free electrons, ions, and radicals, and neutral particles, such as atoms and molecules. Some of these particles are in an excited state and can return to their ground state by photon emission. In plasma, certain electrons are free, allowing positive and negative charges to move somewhat independently from each other. Plasma is a consequence of gases that are excited into energetic states by radio frequency (RF), microwave (MW), or electrons from a hot filament discharge, among other possibilities.

Plasmas are frequently subdivided into nonequilibrium (low-temperature/cold) and equilibrium (high-temperature/hot/thermal) plasmas. High temperatures used in thermal plasmas are destructive for polymers, and typically applications for biopolymer surface modification will make use of cold plasmas. However, in some embodiments, hot plasmas may be used. Low pressure and atmospheric plasma are solvent-free techniques that have been the subject of intense research. Cold plasma allows for the incorporation of a range of desired chemical functions on a large array of surfaces, including relatively chemically inert substrates. Modulating energy, nature of gas used, and treatment time allows for control of the density of deposited chemicals and control of the final surface energy, without changing the mechanical properties of the bulk material. Another advantage is that plasma allows to uniformly coat 3D surfaces, regardless of their geometry.

Atmospheric plasma technology has the advantages of being cheaper, easily scaled up to industrial dimensions and integrated in in-line processes. Nonetheless, it suffers from instabilities in the discharge that lead to thermal discharge that constricts to a narrow current channel, and increases the gas temperature. A pulsed regime, such as dielectric barrier discharges (DBDs), pulsed corona and MW discharges can prevent this transition. Low pressures plasmas operating between 10⁻³-1000 Pa are easier to control as the discharge is more stable but are more expensive than atmospheric plasma technologies.

The present invention is concerned with numerous bioconjugation techniques to immobilize bacteriophages on different substrates, including biodegradable amino-acid based polymers, commercial gauzes, metallic implants, among others. To that effect, the present invention uses low-pressure plasmas, atmospheric plasmas or both low-pressure and atmospheric plasmas in sequence. Low-pressure and atmospheric plasma treatments are usable to directly and indirectly introduce different functionalities on inert surfaces and subsequently immobilize bacteriophages.

In specific examples, cold nitrogen plasma treatment was used for the incorporation of reactive primary amines, among other species, on different surfaces and subsequently immobilize bacteriophages, endolysins and microencapsulated phage formulations. Nitrogen, ammonia and N₂/H₂-plasmas can be used to introduce primary, secondary, and tertiary amines, as well as amides for the immobilization of bacteriophages.

Oxygen plasmas were used to introduce oxygen containing functional groups such as COOH, peroxide, and OH functionalities on different surfaces and subsequently immobilize bacteriophages.

CO₂ or CO-plasmas can also be used to introduce carboxylic groups, and CO₂-plasma treatment can also produce hydroxyls, ketones, aldehydes, and esters that can be used to immobilize phages.

Argon plasmas could be used to introduce free radicals and subsequently attach phages.

Different gases such as Ar, He, O₂, N₂, NH₃, and CF₄ can be used to create, substitute functional groups, or create radicals, on the surface. The introduced functionalities were subsequently used to bind bacteriophages on the surface.

He and Ar plasmas could be used to introduce free radicals on the surface which can produce peroxides and hydroperoxides on the surfaces when exposed to atmosphere or O₂, and can be used to initiate a polymerization reaction.

Plasma treatment can be made with NH₃, N₂/H₂, He, O₂, Ar, N₂, O₂, CO, CO₂, NO, NO₂, SO2, Ne, H₂, air, or a combination thereof, among others, and subsequent coating with phages.

Grafting methods using a heterobifunctional arm are usable to attach the phages covalently on the surfaces, including carbodiimide coupling of a carboxylic group created on the surface with a primary amine on the bacteriophage's capsid. This can be done, for example, done using 1-ethyl-3-(3-(dimethylamino)propyl)carbodiimide hydrochloride (EDC) to activate the cross-linking reaction, and N-hydroxysulfosuccinimide (sulfo-NHS) to stabilize the reaction.

Graft polymerization may be achieved by ionic mechanism, coordination mechanism, and free-radical mechanisms, among others.

Grafting of acrylic acid (AA) by low pressure plasma is usable to immobilize phages. Postirradiation grafting of AA with subsequent carbodiimide mediated immobilization of bacteriophages can be achieved.

Monomers, such as AA, 4-vinylpyridine, and 1-vinylimidazole in the vapor phase can be deposited on the substrate to create a coating on the surface that can be used to subsequently attach bacteriophages.

Plasma polymerization using a precursor monomer (examples: acrylate chemistry, ethyl lactate, lactic acid, e-caprolactone; or a mixture of hydrocarbons with other gases NH3, O2, and CO2, etc.) can be used to give rise to the formation of a thin organic film overcoat that can be tailored for controlled release of the bacteriophages.

Active and remote treatments in atmospheric plasmas can be used to immobilize phages.

Remote plasma treatment is usable for the surface modification of biodegradable polymers in order to enhance radical reaction and restrain electron and ion etching.

Corona discharge, dielectric barrier discharges, RF discharges, electron cyclotron resonance (ECR) plasma reactors, Microplasmas, and plasma jets, such as plasma needles, plasma plumes, plasma pencils, and plasma torches can be used to modify a surface and subsequently immobilize phages. Plasma jets can also be used to prepare a wound to subsequently apply bacteriophages.

Phages can be immobilized by similar methods on medical devices, medical implants such as hip and knee implants and biomaterials, among others.

Gas chemistries for low-pressure plasma immobilizations can be oxygen, argon, helium, nitrogen, ammonia, hydrogen, nitrous oxide, carbon dioxide, air, ethylene, hexafluoropropylene, among others.

Low pressure plasmas can have any suitable pressure, for example about 100 mTor. In other examples, the pressure is between 10 and 1000 mTor, or between 1 and 1000 mTor, among other possibilities.

Liquid chemistries for low-pressure plasma can be methanol, water, allylamine, ethylenediamine, acrylic acid, hydroxymethylmetacrylate, propylethylglycol, hexamethyldisyloxane, aminosilanes, carboxylsilanes, hydroxylsilanes, mercaptosilanes, among others.

We have considered the attachment of bacteriophages to Titanium and stainless steel surfaces, among other possibilities, using plasma polymerization for the prevention and treatment of periprosthetic joint infections. Our results suggested that it is possible to attach bacteriophages to Ti-implants, in a manner that will allow phages to retain their activity and infectivity. Phage treated titanium rods, show a higher efficiency at lysing S. aureus bacteria than non-treated controls of PPE:N treated controls. It is believed that similar results may be obtained with other metals.

In order to improve the biocompatibility and kinetics of the bone formation process, we endeavored to improve the surface properties of the Ti-rods. The literature reports that excessive release of metal ions from the material may be a potentially suppressing effect on bone growth and integration. From a biological point of view, a TiN coating decrease the diffusion of Ti ions from the bulk metal, optimize the surface properties, and reduces the negative effects, which may be of potential interest when implanted in vivo.

The literature also reports on the biological reactions at TiN surfaces, including the responses of bone, soft tissues, blood, platelets, human mesenchymal stem cells, and osteoblasts. Several of those studies indicate that TiN surfaces have beneficial or comparable properties in comparison with other currently and frequently used materials.

We hypothesized that a Ti-PPE:N+bacteriophages would improve bone growth and integration, as well as suppress commonly associated infections, such as the one caused by multi-drug resistant Staphylococci, Pseudomonas, and Klebsiella species.

Many of the methods discussed herein have been explored and used in the context of small molecule-loaded materials, however bacteriophages present certain challenges that have not, thus far, been solved. As biological organisms, bacteriophages exhibit many of the inherent drawbacks of these systems, most notable lower stability and short shelf life. Many methods are possible for extending and protecting their lifespan and efficacy including lyophilization and microencapsulation. Since these materials will be used in the treatment of possibly septic areas, the products themselves must also be sterile, which presents its own challenges, once again due to the biological nature of the phages.

Polymers, such as poly(ester urethane urea)s (PEUR), and other block co-polymer mentioned in the above-referenced PCT application, can be formulated in many ways, for example solid polymer layer, porous polymer layer, and microcapsule-bearing layer on the surface of orthopedic implants and other medical devices to prevent, or at least reduce, surface formation of bacterial biofilms.

In some embodiments, we propose to deposit a thin (typically, up to some hundreds of nanometers), strongly-adhering plasma polymerization coating, which is applied to orthopedic implants. The strong adherence derives, in some embodiments, from the fact that, by the very nature of the plasma process, the coating is covalently bonded to the substrate surface.

Experience has shown that plasma polymer coatings can be made in such a way as to manifest enormously large effective surface areas, a distinct advantage in this context: The obvious reason is that a large amount of phages can then be incorporated per unit area (e.g. per square centimeter) of substrate surface.

For this project, different gas mixes (carbon rich, nitrogen rich, etc.) and percentages were tested.

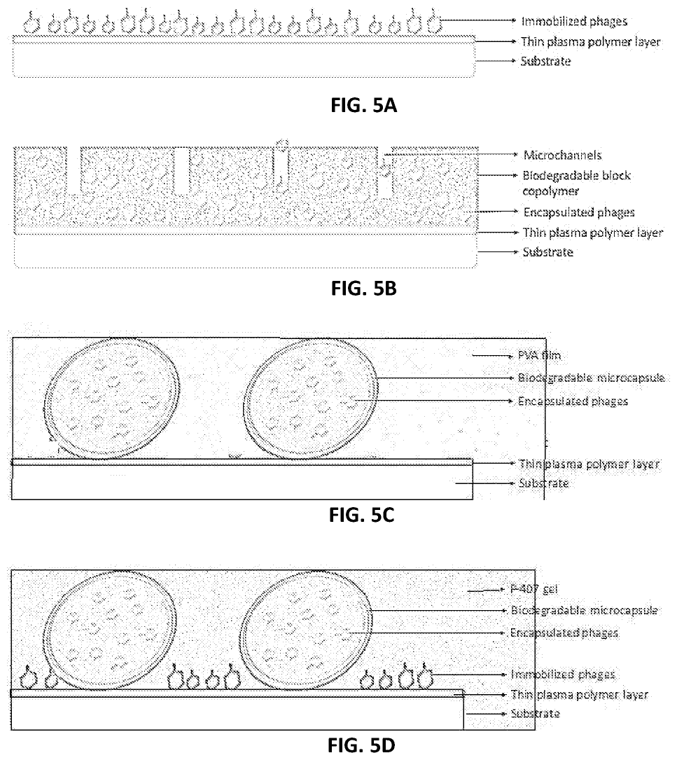

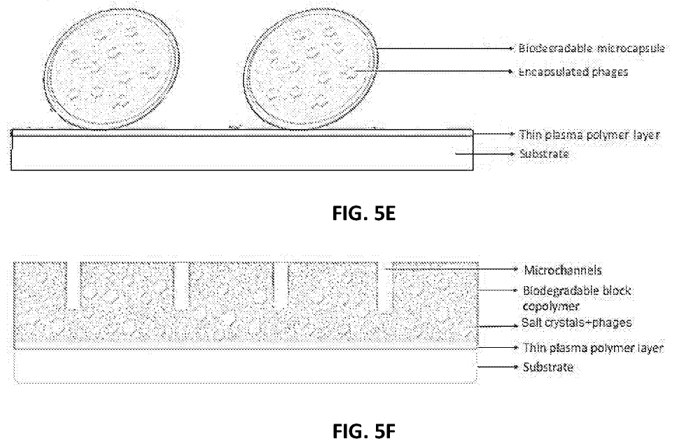

Three exemplary embodiments of microcapsules-containing formulations on the plasma-treated orthopedic implant are illustrated in FIG. 5 1) direct immobilization of bacteriophages on the thin plasma polymer layer; 2) the second strategy consist on dip-coating the implant in a block copolymer containing bacteriophages following plasma surface activation. This coating is shown to possess many micro-channels that allow easy transport of bacteriophages to the top outer surface, where some portion of the phages is required immediately. These micro-channels can be created by leaching of incorporated salt crystals, among other possible approaches; 3) the third strategy consists of spraying the implants with a spray patch formulation (microencapsulated phages). The principle virtue of this method will be to protect the underlying bacteriophages and microspheres against possible removal or other damage during handling, packaging, implantation by the surgeon, etc.

Two approaches may be used for the attachment of polymers to the surfaces of the implants: covalent attachment, and non-covalent attachment.

In a first example, microspheres are fabricated using a water-in-oil-in-water double emulsion followed by solvent evaporation. The first water in oil emulsion will be made by homogenizing a 1% poly(vinyl alcohol) (PVA) solution with newly synthesized polymers dissolved in organic solvent or a mixture of solvents, using a high-speed homogenizer with a 10 mm dispersing element. This first emulsion will be added dropwise to the bacteriophage cocktail containing 2% of PVA, thus forming a water-in-oil-in-water emulsion. Solvent will be left to evaporate. PVA or pluronics will be then added to obtain a spray patch formulation. The sprays can be sprayed on the implant before its insertion in the human body or after the implant is inserted. The spray can also be used (sprayed) on the adjacent tissues to the implant before or after fixation of the implant.

For example, for covalent polymer attachment to the surface, a polymer including monomers consisting alkyl diester amines with pendant carboxylate groups may be used. These can be made using a protected glutamic acid as the amino acid reagent. Using a suitable polymerization, using triphosgene and diamines, we can then generate a polymer with pendant carboxylate groups that can be activated with standard coupling agents (DCC or HOBt) to easily attach them to surface bound amine groups or other groups of the plasma polymer layer. Otherwise, rather than using the polymer as an electrophile (with the carboxylic acid), the polymer can also act, for example, as a nucleophile by using cysteine for the diester monomer. After polymerization, the pendant thiol can then be used to participate in a thiol-ene click reaction to covalently attach to surface-bound alkene groups.

For electrostatic surface attachment, there are two possibilities: positive surface-negative polymer, or negative surface-positive polymer. For example. surfaces may be treated with nitrogen containing plasma to yield surfaces with have a positive charge due to the primary, secondary, and tertiary amines as well as the imine groups. To interact with these positive charges, polymers such as glutamic acid-containing polymers may be used without activation, such that they are negatively charged at physiological pH, and can therefore interact with the positive charges of the plasma polymer. In another example, cysteine containing polymers can also be used, where after a peroxide mediated oxidation, they are transformed to strongly negative sulfonate groups, which can strongly interact with positive surface charges on the plasma polymer. Negatively charged plasma polymer surfaces may be produced using oxygen or sulfur containing plasma polymers, for example, due to the resulting carboxylic acids and thiols, respectively. To interact with these negative charges, (positively charged) arginine-containing monomers and polymers can be used, among other possibilities. Non-limiting examples of polymers including amino acids suitable for use in electrostatic binding are provided in the summary of the invention.

The above suggests a medical device comprising a substrate defining a surface; a plasma polymer layer bound to and coating the surface; and a bactericide layer bound to the plasma polymer layer, the plasma polymer layer being between the substrate and the bactericide layer. The substrate is a portion of the medical device to cover with the plasma polymer, and may represent part or the entire medical device. The medical device may or may not have moving parts.

The medical device is any device intended to contact human or animal tissues or fluids of a subject. The medical device may or may not have pharmaceutical properties. For example, the medical device is intended to be permanently or temporarily implanted in a human or animal. A non-limiting example of such a medical device would be an orthopedic implant. The substrate is a portion of the medical device on which a plasma polymer will be deposited. Typically, such a portion is exposed to the cells and/or fluids of the subject.

The bactericide layer is a layer that has some bactericide properties. The bactericide layer helps in reducing or preventing bacterial growth adjacent to the bactericide layer and on the bactericide layer. The bactericide layer achieves its bactericide properties at least in part by incorporating bioactive bacteriophages, bacteriophage related products or both bioactive bacteriophages and bacteriophage related products. Examples of bacteriophage related products include endolysins, lysostaphins, phage proteins, phage enzymatic formulations, and combinations thereof. An antibiotic could also be added to the bactericide layer.

Generally speaking, the surface of the substrate of the medical device is coated with the bactericide layer by first exposing the surface to a plasma to form a plasma polymer layer bound to the surface and then binding a bactericide layer to the plasma polymer layer. The plasma polymer in the context of the present document is therefore a polymer formed by plasma polymerization.

The plasma is for example a cold plasma, either at a pressure below atmospheric pressure, or an atmospheric pressure plasma. The plasma may include one or more of NH₃, N₂/H₂, He, O₂, Ar, N₂, O₂, CO, CO₂, NO, NO₂, SO₂, Ne, H₂, air and CF₄, depending on the exact chemistry required to attach the bactericide layer. The plasma may also include monomers in some embodiments, for example cetic acid, 4-vinylpyridine, 1-vinylimidazole, an acrylate, ethyl lactate, ethylene, lactic acid, e-caprolactone, methanol, water, allylamine, ethylenediamine, acrylic acid, hydroxymethylmetacrylate, propylethylglycol, hexamethyldisyloxane, aminosilanes, carboxylsilanes, hydroxylsilanes and mercaptosilanes. The plasma process, in some embodiments, forms reactive groups at the surface of the plasma layer. Examples of such reactive groups include: free radicals, COOH, peroxide, OH, primary amines, secondary amines, tertiary amines, amides, carboxylic groups, hydroxyls, ketones, aldehydes, esters and combinations thereof

Examples of suitable compositions for substrates include metal and metal alloys, such as non-limitingly iron, copper, zinc, lead, titanium, aluminium, titanium, gold, platinum, silver, cobalt, chromium, vanadium, tantalum, nickel, magnesium, manganese, cobalt chrome, nickel titanium, titanium vanadium aluminium, and stainless steel. Other suitable compositions for substrates include polymers.

The plasma polymer layer can have any suitable thickness. In some embodiments, the plasma polymer layer is between 10 and 1000 nm thick. In other embodiments, the plasma polymer layer is between 100 and 500 nm thick.

The bactericide layer may have any suitable composition. The plasma polymerization process forms the plasma polymer layer and leaves the plasma polymer layer activated. In embodiments in which this activation is gradually lost, for example through reaction with ambient air, addition of the bactericide layer may be performed quickly enough that this activation is not lost. In other embodiments, the activation of the plasma polymer layer is not lost or loss of this activation is not critical. This latter possibility would occur if the bactericide layer includes reactive group that can react with the deactivated plasma polymer.

In a first example, with reference to FIG. 5A, the bactericide layer includes bioactive bacteriophages directly bound to the plasma polymer layer, for example through covalent bounds. In this embodiment, the bacteriophages are not embedded in any material. The bacteriophages are provided outside of the plasma polymer layer and are exposed to the environment. FIG. 5A illustrates a medical device in accordance with the invention in a very schematic form as the present invention is usable in a wide variety of medical devices having different shapes and dimensions.

To attach the bacteriophages to the plasma polymer, one can contact a suspension including the bacteriophages with the plasma polymer layer. In some embodiments, a linking agent that promotes covalent bonding between the bacteriophages and the plasma polymer is also provided.

In a second example, with reference to FIG. 5B, the bactericide layer includes a coating material in which bacteriophages are dispersed. In a variant of this second example, the bacteriophages are replaced or complemented by phage-related products dispersed in the coating material. The coating material is selected to release the bacteriophages when the medical device has been implanted. This release may be relatively quick, if the coating material is easily dissolved in the subject, or it can be relatively slow, if the coating material is only slowly degraded in the subject.

It should be noted that in some embodiments, not shown in the drawings, bacteriophages and/or phage-related products can be also bound to the surface of the coating material, for example using plasma treatment of the coating material. In such embodiments, the surface bacteriophages can provide a first relatively high bactericide activity present as soon as the implant is implanted, while the deeper bacteriophages, contained in the coating material are released slowly over a period of days, months or years to maintain a smaller bactericide activity.

The coating material may be covalently or electrostatically bound to the plasma polymer layer. Examples of suitable coating materials include non-limitingly the polymers described the summary of the invention. However, any other suitable polymer that may bind with the plasma polymer and that can maintain the bioactivity of the bacteriophages and/or phage-related products may be used.

One method of achieving the bactericide layer in this example includes dip-coating the plasma polymer covered substrate in a polymer solution in which the bacteriophages and/or phage-related products are suspended. After evaporation of the solvent, a relatively durable polymer layer is then formed, the bacteriophages and/or phage-related products being embedded in the polymer layer. However, any other suitable method of contacting the polymer solution with the plasma polymer, such as spray-coating, among others, is within the scope of the invention.

In some embodiments, the coating material is a polymer defining an exposed surface and the bactericide layer defining microchannels extending in the coating material from the exposed surface. Such microchannels can facilitate release of the bacteriophages and/or phage-related products. They can be formed in any suitable manner, for example by leaching salt crystals embedded in the coating material (that would be present in the solution containing the coating material) or by plasma etching the coating material after it has coated the plasma polymer. The salt crystals may leach in-vivo, after implantation of the implant.

In a third example, as seen in FIG. 5C, the bactericide layer includes bacteriophage-containing biodegradable microcapsules embedded in a coating material, here denoted as PVA, the coating material being bound to the plasma polymer. An example of such microcapsules is described in PCT application PCT/IB2017/053744 filed Jun. 22, 2017, but other microcapsules are within the scope of the invention. Examples of suitable coating materials in these embodiments include Poloxamer 407 and polyvinyl alcohol (PVA), among other possibilities. Such a bactericide layer can be made by spray-coating the plasma polymer with a suspension of the microcapsules in the coating material or by dipping the plasma polymer in the suspension of microcapsules.

In a fourth example, as seen in FIG. 5D, the bactericide layer includes both bacteriophages and/or phage-related products bound to the plasma polymer, as in the first example above, and the bacteriophage-containing biodegradable microcapsules embedded in a coating material, the coating material, here denoted as P-407, being bound to the plasma polymer, as in the third example above. An example of such microcapsules is described in PCT application PCT/IB2017/053744 filed Jun. 22, 2017, but other microcapsules are within the scope of the invention. Examples of suitable coating materials in these embodiments include Poloxamer 407 and polyvinyl alcohol (PVA), among other possibilities. Such a bactericide layer can be made by spray-coating the plasma polymer with a suspension of the microcapsules in the coating material. In yet other embodiments, the suspension of microcapsules also includes suspended bateriophages, not contained in the microcapsules.

In a fifth example, as seen in FIG. 5E, the bactericide layer includes bacteriophage-containing biodegradable microcapsules bound to the plasma polymer, either covalently or electrostatically. Manufacturing such a bactericide layer requires that the biodegradable microcapsules be in contact with the plasma polymer. This can be achieved either by contacting a powder including the biodegradable microcapsules with the plasma polymer, or by contacting a suspension in a liquid of the biodegradable microcapsules with the plasma polymer. Linking agents promoting reaction of the microcapsules with the plasma polymer may be used in some embodiments.

In a sixth example, as seen in FIG. 5F, the bactericide layer includes salt crystals on which bacteriophages are adsorbed. This example is similar to the variant of the second example above. Such salt crystals with adsorbed bacteriophages are described in the above-referenced PCT/IB2016/001006 application. In this example, additional bacteriophages may or may not be present in the coating material.

Examples of suitable salts include calcium salts, magnesium salts, strontium salts, and barium salts, for example calcium carbonate, calcium phosphate, magnesium carbonate, and magnesium phosphate. In some embodiments, the salt is a mixture of MgCO₃ and CaCO₃. In some embodiments, the weight ratio of MaCO₃ to CaCO₃ ranges from 5:95 to 95:5, such as the ratio is 5:95. The at least one inorganic salt such as calcium and magnesium salts may positively influence wound healing by stabilizing and activating the bacteriophage.

Briefly, preparation of the salt crystals with adsorbed bacteriophages includes: mixing and holding (incubating) at least one inorganic salt and at least one bacteriophage, filtrating the suspension obtained to produce the at least one bacteriophage adsorbed (immobilized) wet solid product, washing the obtained wet solid product with saline solution optionally; and drying the obtained wet solid product through vacuum drying, freeze drying or spray-drying to obtain a first composition.

Incorporation of the salts in the coating material is performed as follows in some embodiments, but other manufacturing methods are within the scope of the invention. A bactericide layer is prepared as follows: a. mixing the first composition described above with a mixture comprising an organic solvent and at least one polymer, such as the polymers disclosed hereinabove; b. casting the resulting mixture from step (a) onto the plasma polymer; and c. evaporating the organic solvent to obtain the bactericide layer.

Alternatively, provided is a process for preparing the bactericide layer, comprising a. mixing a liquid comprising at least one bacteriophage with a mixture comprising an organic solvent and at least one polymer, such as the polymers disclosed hereinabove; optionally adding at least one filler selected from the inorganic salts disclosed above and at least one another bioactive agent; b. casting the resulting mixture from step a onto the plasma polymer; and c. evaporating the organic solvent to obtain the bactericide layer.

In some embodiments, the organic solvent is chloroform. In some embodiments, the mixture comprising an organic solvent and at least one polymer further comprises additional bioactive agent chosen from antiseptics, antiinfectives, such as bacteriophages, antibiotics, antibacterials, antiprotozoal agents, and antiviral agents, analgesics, anti-inflammatory agents including steroids and non-steroidal anti-inflammatory agents including COX-2 inhibitors, anti-neoplastic agents, contraceptives, CNS active drugs, hormones, hemostatics, and vaccines.

In some embodiments, the coating material comprises poly (ester amide urea), at least one or more bacteriophage, calcium carbonate, magnesium carbonate, benzocaine, ciprofloxacin, and chymotrypsin

In some embodiments, the at least one salt is selected from inorganic salts as disclosed herein. In some embodiments, the at least one salt and at least one bacteriophage in the form of liquid is mixed in an appropriate w/v (g/mL) ratio such as a ratio of 1:10. In some embodiments, the process for preparing the first composition is carried out at room temperature and under sterile conditions.

In some embodiments of the coating material, the at least one polymer is selected from poly (ester amide urea), poly (ester urethane urea), poly (ester amide urethane urea), and poly (ester amide urethane), as described in the Summary of the Invention section.

In all the above examples, the bactericide layer may also include one or more of the following bioactive agents antiseptics; anti-infectives such as bacteriophages, antibiotics, pain relievers, antibacterials, antiprotozoal agents, and antiviral agents; analgesics; anti-inflammatory agents including steroids and non-steroidal anti-inflammatory agents including COX-2 inhibitors and anti-neoplastic agents; contraceptives; CNS active drugs; hormones; enzymes; hemostatics; and vaccines. Non-limiting examples of the enzymes include those that can catalyze the hydrolysis (erosion) of the polymer disclosed herein. The hydrolysis (erosion) of the polymers disclosed herein can be important for the release of the at least one bioactive agent into the surrounding tissues. The at least one enzyme may also be used, as a non-limiting example, to treat wounds and abrasions by removing the dead or infected skin from the site of injury. Non-limiting examples of the at least one enzyme include papain, collagenase, elastase, fibrinoylsine, hyaluronidase, trypsin, α-chymotrypsin and lipase. In some embodiments, the at least one enzyme is selected from trypsin, α-chymotrypsin and lipase. Non-limiting examples of such antibiotics include fluoroquinolones (e.g., tetracycline, ciprofloxacin, and levofloxacin), monoxycarbolic acid antibiotics (e.g., mupirocin), aminoglycosides (e.g., neomycin), macrolide antibiotics (e.g., erythromycin), bacitracin, polymixyin, and mixtures thereof. Exemplary pain relievers include, but are not limited to, benzocaine, lidocaine, tetracaine, pramocaine, dibucaine, and mixtures thereof.

Various embodiments of the invention include a bactericide layer that includes bacteriophages or bacteriophage-related product, or both, referred to as “the bactericide” in the present paragraph. The bactericide may be directly bound to the plasma polymer layer, dispersed in a coating material bound to the plasma polymer layer, embedded in microcapsules, which can be directly bound to the plasma polymer layer or dispersed in the coating material, directly bound to the coating material, outside of the coating material, or adsorbed on salt crystals embedded or otherwise bound to the coating material. All suitable combinations of these binding modalities for the bactericide are also within the scope of the invention. In some embodiments, the bactericide layer may have a composition or other properties that changes as a function of the distance from the plasma polymer layer.

It should be noted that the plasma polymer may or may not be entirely covered by the bactericide layer, either through imperfections in manufacturing or by design.

### EXAMPLES

### Example 1: Immobilization of Phages on Co-Polymer Surfaces

Low pressure plasma using 4 different chemistries (COOH gas, NH₃ gas, NH₃ vapor, and COOH vapor) were tested, along with 2 chemistries for atmospheric pressure plasma (Room air, N₂ gas). For human applications, we tested immobilization and activity of immobilized phages on an amino-acid-based-biodegradable-copolymer. The results were positive for all chemistries. We also tested the immobilization of phages on commercial gauzes. The experiments that were performed are summarized in Table 1.

TABLE 1
Immobilization of phages on polymer and gauze coupons following plasma treatment.

| | | | | | | Atmospheric | | |
|---|---|---|---|---|---|---|---|---|
| Sample | Low pressure | Low pressure | Low pressure | Low pressure | Low pressure | pressure | Atmospheric | |
| Name | COOH gas | NH₃ gas | NH₃ vapour | COOH vapour | Acrylate vapour | Room Air | pressure N₂ | Bacteria |
| PEAU | No lysis | No lysis | No lysis | No lysis | No lysis | No lysis | No lysis | SaX |
| TMN CTL | No lysis | No lysis | No lysis | No lysis | No lysis | No lysis | No lysis | SaA12 |
| PEAU | Lysis | Lysis | Lysis | Lysis | Lysis | Lysis | Lysis | SaX |
| TMN | Lysis | Lysis | Lysis | Lysis | Lysis | Lysis | Lysis | SaA12 |
| Phage | | | | | | | | |
| treatment | | | | | | | | |
| Gauze 1 | No lysis | No lysis | No lysis | No lysis | No lysis | No lysis | No lysis | SaX |
| CTL | No lysis | No lysis | No lysis | No lysis | No lysis | No lysis | No lysis | SaA12 |
| Gauze 1 | Lysis | Lysis | Lysis | Lysis | Lysis | Lysis | Lysis | SaX |
| Phage | Lysis | Lysis | Lysis | Lysis | Lysis | Lysis | Lysis | SaA12 |
| Treatment | | | | | | | | |
| Gauze 2 | No lysis | No lysis | No lysis | No lysis | No lysis | No lysis | No lysis | SaX |
| CTL | No lysis | No lysis | No lysis | No lysis | No lysis | No lysis | No lysis | SaA12 |
| Gauze 2 | Lysis | Lysis | Lysis | Lysis | No lysis | No lysis | No lysis | SaX |
| Phage | Lysis | Lysis | Lysis | Lysis | No lysis | No lysis | No lysis | SaA12 |
| Treatment | | | | | | | | |
| Gauze 3 | No lysis | No lysis | No lysis | No lysis | No lysis | No lysis | No lysis | SaX |
| CTL | No lysis | No lysis | No lysis | No lysis | No lysis | No lysis | No lysis | SaA12 |
| Gauze 3 | Lysis | Lysis | Lysis | Lysis | Lysis | No lysis | Lysis | SaX |
| Phage | Lysis | Lysis | Lysis | Lysis | Lysis | No lysis | Lysis | SaA12 |
| Treatment | | | | | | | | |

Each sample type went through all the 7 plasma conditions as referred to (1) Low pressure COOH gas; (2) Low pressure NH₃ gas; (3) Low pressure NH₃ vapor; (4) Low pressure COOH gas; (5) Low pressure acrylate vapour; (6) Atmospheric pressure Room Air; and (7) Atmospheric pressure N₂ gas.

Low pressure plasma setup were 100 mTor, at 13.56 MHz. NH₃ vapour refers to the vaporization of allylamine. Surface modification by plasma treatment were performed as follows. For low pressure plasma, PEAU (Polyester amide urea (PEAU), a leucine-based poly ester amide polymer) polymer films and commercial gauzes were cut into 2 cm×2 cm squares. Plasma treatment was performed in a 3.5 ft³ square chamber with 9 removable shelves on 1.5 inch centres and side wall electrode configuration. The uniform glow discharge was connected to a radio frequency generator. Samples were treated for 1 minute under a power of 100 W and a vacuum of 100 mTorr. Atmospheric plasma was created with compressed air supplied at a pressure of 3 atm and a flow rate of 100 L/h, and a working distance of 10 mm from the samples. Co-polymer and gauze samples sides were immobilized on microscope glass slides; This allowed the production of free radical groups, coupled with active species from the plasma environment to form polar groups such as —(C—O)—, —(CO)— and —(C)—O— on the substrate surface permitting subsequent phage adhesion.

The treatment with liquid phages consisted in immersing the samples, after plasma treatment, in a liquid solution of staphylococcal bacteriophages (10⁹ PFU/mL) overnight, and then washing with PBS 3 times. A non-treated control of each sample type was also used. Thus, every sample treated with every single condition plus phages possesses two controls: one non-treated and one treated with plasma only.

Samples were tested on a non-pathogenic bacterium (Staphylococcus xylosus) and a pathogenic bacterium (Staphylococcus aureus), on petri dishes with Staph specific agar. Samples were allowed to dry under the biological hood prior to analysis.

Samples were placed on Petri dishes, preliminary toped with Staphylococcus specific agar and bacteria, and incubated overnight at 37° C. Lysis and samples diameter were measured for each sample for each condition. A picture of each plate was taken.

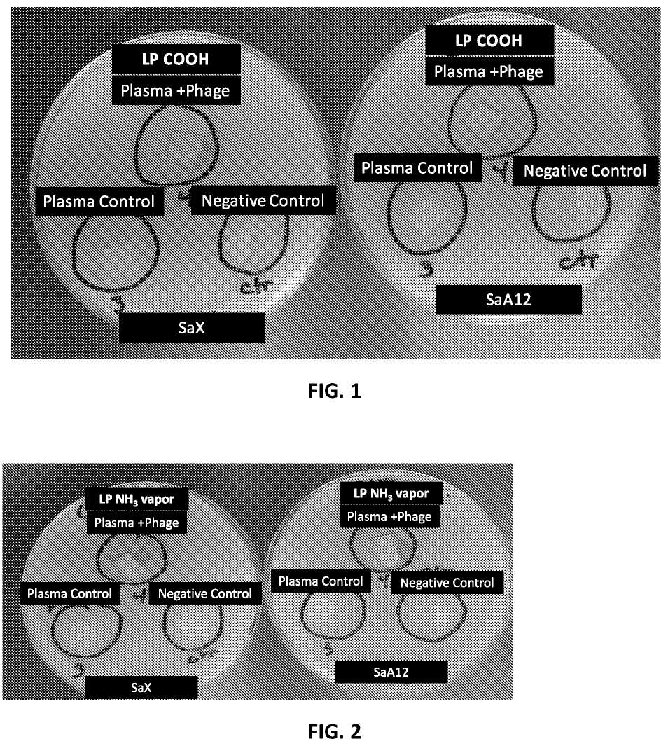

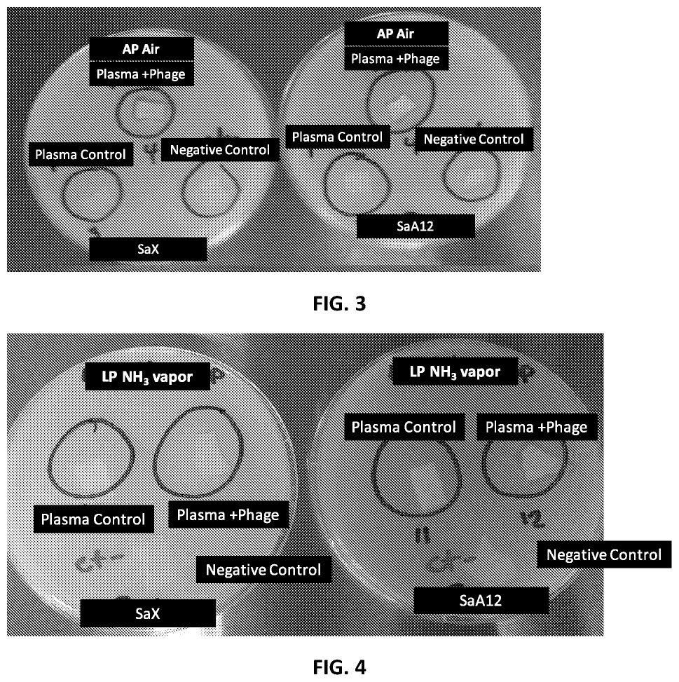

FIGS. 1 to 3 are representative pictures of petri dishes used to determine success or failure of immobilization after overnight incubation. Table 1 summarizes sample and lysis dimensions for these petri dishes. “No Lysis” denotes no significant lysis, and therefore unsuccessful attachment of phages, and “lysis” denotes successful attachment of phages as shown by a lysis zone around the coupons. LP=Low Pressure and AP=atmospheric pressure. All studies plasma treatments followed by phage attachment were successful for the PEAU coupons.

PEAU is a proprietary amino acid based co-polymer, described in further details in the summary of the invention. PEAU was used as an example of immobilizing phages by plasma on polymer-coated medical devices. PEAU+TMN is the amino acid based co-polymer to which a buffer solution (TMN) not containing phages was added to provide a true negative control. PEAU containing phages is the co-polymer to which phages were incorporated before plasma treatment. This constituted a control to determine if the plasma treatment could affect the phages contained in the copolymer, i.e. inactivate them. The results demonstrated that the plasma treatment did not affect the activity of the phages. PEAU is only one example of a polymer usable with the invention and the present approach can work with many other types of polymer-coated medical devices.

### Example 2: Immobilization of Phages on Commercial Gauze

The effect of plasma polymerization and subsequent phage immobilization on commercial gauzes were studied. Protocols and treatments were as described in the previous section “Example 1”. Gauze 1 is a Johnson and Johnson non-adherent Band-Aid gauze, Gauze 2 is a Johnson and Johnson Band-Aid gauze pads with Quiltvent technology, Gauze 3 is a Fomedica gauze made of 70% rayon and 30% polyester.

Results are summarized in Table 1 wherein “Lysis” denotes successful phage immobilization as demonstrated by the lysis of S. aureus and S. xylosus strains, and “No lysis” denotes unsuccessful immobilization. FIG. 4 is a representation of the petri dishes on which activity of the bacteriophages was evaluated.

### Example 3: Immobilization of Phages on Miniature Orthopaedic Implants

Surface prior and following immobilization of bacteriophages were monitored by field-emission scanning electron microscopy (FEG-SEM, operating at 5 kV). Peak force tapping mode AFM measurements were performed using Scan Asyst-Air tip.

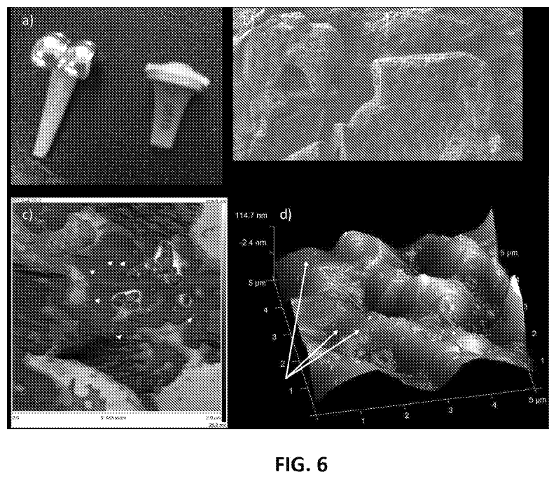

Plasma is generated with a stainless steel electrode. Chamber was cleaned before arrival. Pre-deposition was made with treatment 1 consisting of 10 sccm ammoniac and 10 sccm ethylene. Pressure was 80 Pa, at 20 W. Samples, petri dishes, sterile tweezers were inserted in the box via the chamber. The miniature implants were placed on a petri dish so they don't touch each other. Petri dish was positioned on the electrode. Treatment 1, as described above, was applied for 10 minutes in order to achieve a 100 nm plasma polymer deposit on the surface. Samples were subsequently immersed in a bacteriophage solution for one hour at room temperature. The samples were then washed 3 times with deionized water, and dried under the hood. FIG. 6a shows a miniature knee implant treated in this fashion. SEM images show the high porosity of the sample (FIG. 6b). AFM analysis was used to investigate the relationship between surface topology and biological interactions. FIG. 6d shows the covalent immobilization of phages on the surface of the implant following plasma treatment.

### Example 4: Immobilization of Phages on Titanium Rods (Atomic Force Microscopy)

Plasma is generated with a stainless-steel electrode. Chamber was cleaned before arrival. Pre-deposition was made with treatment 1 consisting of 5 sccm ethylene and 20 sccm carbon dioxide. Pressure was 80 Pa, at power was 20 W. Samples, petri dishes, sterile tweezers were inserted in the box via the chamber. The miniature implants were placed on a petri dish so they don't touch each other. Petri dish was positioned on the electrode. Treatment 1, as described above, was applied for 10 minutes in order to achieve a 100 nm thick plasma polymer deposit on the surface

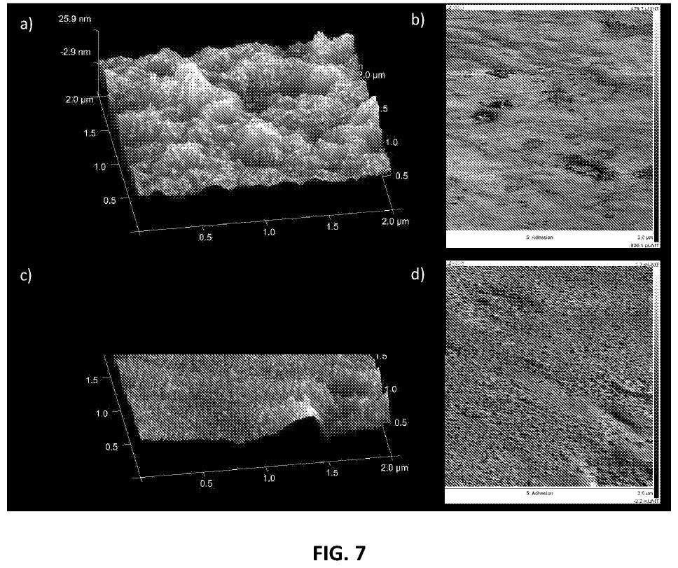

Topological images show globular adhesions of 30 to 50 nm (FIG. 7, panel c) protruding from the implant surface (FIG. 7, panel d) corresponding to the bacteriophage's heads. Height sensor AFM images of untreated rods show large vacancy islands (FIG. 7, panel a) which clearly contrasts with treatment samples, in which vacancy islands seem to be uniformly filled with bacteriophages (FIG. 7, panel c). Furthermore, untreated (FIG. 6, panel b) and treated (FIG. 7, panel d) samples have noticeably different topological image with compact globular bodies of approximately 50 nm in diameters (corresponding to phages) finely distributed on the titanium rod, covering the substrate surface almost completely.

### Example 5: Immobilization of Phages on Titanium Rods (Microbiological Determination of Activity and Potency)

### Example 5 a: Plaque Assay

Plasma is generated with a stainless steel electrode. Chamber was cleaned before arrival. Pre-deposition was made with treatment 1 consisting of 10 sccm ammoniac and 10 sccm ethylene. Pressure was 80 Pa, at 20 W. Rods, petri dishes, sterile tweezers were inserted in the box via the chamber. Rods were placed on a petri dish so they don't touch each other. Petri dish was positioned on the electrode. Treatment 1, as described above, was applied for 10 minutes in order to achieve a 100 nm thick plasma polymer deposit on the surface. After treatment, rods were put back in 2 mL glass vials containing either a solution of phages in TMN buffer or a solution of endolysins in PBS buffer. Control groups were either incubated in TMN or PBS without the antibacterial, and sealed in a bag to preserve from 02 contamination. samples were rinsed 3 times with SuperQ water, dried for 2 hours under the hood before further testing.

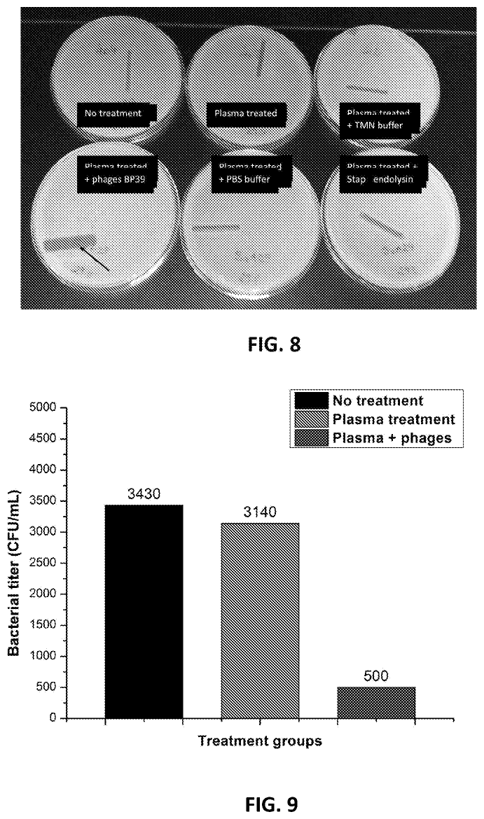

We performed plaque assays to assess the capture of host cells by phages, and assess if the phage particles retained their active conformation. The rods were directly put into contact with a bacterial (SaA29) top agar to assess direct lysis. SaA29 is a methicillin resistant Staphylococcus aureus and the natural host for bacteriophage BP39, a podoviridae. The plaque assay (FIG. 8) clearly shows that only the titanium rod treated with BP39 bacteriophages and S. aureus endolysins were able to lyse the bacterial lawn. Covalently immobilized bacteriophages and lysins thus retained their infectivity.

### Example 5 b: Attachment Assay

The colonization attachment assay provides a method of quantifying the number of bacteria that adheres to coupons following inoculation with bacteria. This method describes the steps necessary to quantify the amount of Staphylococcus aureus that adheres to test coupons. The test coupons were treated with plasma as described in Example 5a, and were incubated with an initial inoculum concentration for attachment in a standard liquid assay. After the 1 hr period, the coupons are washed and used in subsequent steps to determine CFU/pin and in the proliferation assays. An overnight bacterial inoculum of Staphylococcus aureus at a concentration of 10⁸ CFU/mL in 20 mL commercial TSB is prepared. The concentration of bacteria is adjusted to 10⁴ CFU/mL using 0.3% TSB. Pins in a 6 well culture plate. Clean the control pins in sterile/ultrapure deionized water and then autoclave for 15 min at 121° C. 5 mL of inoculum is added into each well and placed in the shaker-incubator at 37° C. and 60 RPM for 1 hr. Inoculum is aspirated from the well and replaced with 5 mL of 1×PBS. Samples are washed 4 times. The pins are transferred to 15 mL centrifuge flip tubes containing 3 mL of neutralization buffer and vortexed for 10 s. The tubes are sonicated for 15 min at 9.4 W in a chilled water bath set to 4° C. and vortexed again for 10 s at the highest speed setting. The pins are removed from the vial and rinsed down into the tube with 2 mL of neutralizing buffer. The washed pins are rolled onto the surface of a TSA-L plate by pressing down with a tweezer in one direction. The vials are centrifuged at 3000 RPM for 5 min at 4° C. 100-200 uL of supernatant and pellet are pipetted in the tube to mix, and plated onto the surface of a TSA-L plate. The plate is incubated for 18-24 hours and the number of colonies on the plate are counted.

There was no significant effect of plasma treatment on bacterial adhesion when compared to the no treatment group. However, we saw a clear reduction in bacterial count for the phage treated group when compared to both controls (FIG. 9), with a 0.8 log reduction in bacterial counts.

### Example 5c: Liquid Proliferation Assay

This method describes the steps necessary to quantify the amount of Staphylococcus aureus that proliferates onto the test coupons following the attachment assay. The test coupons are incubated with an initial inoculum concentration described in the attachment assay. After the 1 hour period, the coupons are washed and allowed to proliferate and used in subsequent steps to determine CFU/pin and in the proliferation assays. An initial inoculum is used for attachment. Pins rock and incubate for an hour. Then, the pins are gently washed and allowed to proliferate for an additional 18-24 hours in soft agar as a simulation of soft tissue and in a standard liquid assay for an additional 18-24 hours after which time, the CFU/pin is determined.

Plates for titration were not TSA-L but Staph specific agar. Bacteria was SaA3 (S. aureus ATCC 25923). CTRL sample is autoclaved 15 min at 121° C. Inoculum of bacteria was titrated at 0.7·10⁴ CFU/mL before incubation, and at 1.1·10⁵ CFU/mL after 1 h incubation.

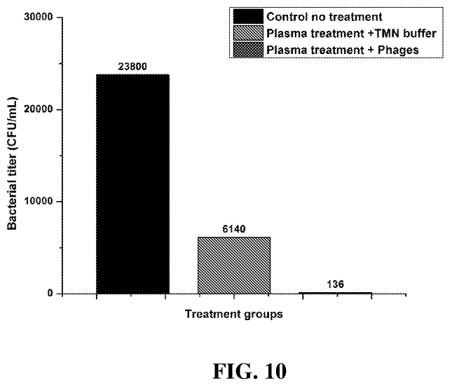

In this assay, we find that there is a significant difference between the no-treatment control group and the plasma treated group. We hypothesize that the plasma treatment affected the hydrophobicity of the rod, thus impacting the ability of bacteria to grow on its surface (FIG. 10).

Moreover, we find a significant difference between both control groups and the phage treatment group, with a 1.65 log reduction when the phage treatment group is compared to the plasma treatment, and a 2.24 log reduction when the phage treatment group is compared with the no-treatment control.

We have successfully immobilized bacteriophages on titanium rods using plasma technology as shown by AFM. Plaque assays show that the immobilized phages retain their activity and infectivity following treatment. Attachment and proliferation assays show a significant difference between bacterial adhesion on control rods when compared with phage immobilized titanium rods with a log reduction of 0.8 and varying between 1.7 and 2.2 for both assays.

### Example 6: Tuning the Surface Properties of Oxygen-Rich and Nitrogen-Rich Polymers

In this work, a set of low-pressure plasma co-polymerized films, prepared from binary gas mixtures of ethylene (C₂H₄)/ammonia (NH₃), and ethylene/carbon dioxide (CO₂) were produced in order to deposit either nitrogen rich or oxygen rich coatings with adjustable chemical composition and surface charge.

A low-pressure capacitively coupled radio-frequency glow discharge in gas mixtures containing a source of carbon and a source of heteroatom, was used to produce the plasma polymerized coatings. The films were deposited on titanium coated k-wire substrates. The substrates were sonicated first in isopropanol (99.9%, Fischer Scientific) and second in super-Q water before being dried under a laminar hood and autoclaved. The titanium rods were mounted on a home-made support inside the plasma chamber.

The depositions were performed in a cylindrical stainless steel vacuum chamber (20 cm in diameter and 50 cm in height) with a disc shaped powered electrode (Ø=10 cm) onto which the samples were placed (FIG. 16).

A showerhead gas distributer, positioned 4 cm above the powered electrode also served as grounded electrode. The chamber is evacuated using a combination of rotary-vane and turbo-molecular (pumps to high vacuum. The processes mixtures are introduced via mass-flow controllers. The gas flow ratio is defined as R=(flow of heteroatom source gas)/(flow of hydrocarbon source gas).

Table 2 contains a description of the various experimental parameters used in the present study. The pressure was maintained constant at 80 Pa during deposition runs by a throttling gate valve. An automatic impedance matching network is used to generate the capacitively coupled radio frequency (RF, 13.56 MHz, Cesar) discharge at a power of 20 W. A typical deposition run lasted about 10 min to deposit coatings around 100 nm thick.

TABLE 2
Gas flow ratios used in deposition of plasma polymer coatings

| Plasma | Gas flow | Hydrocarbon | Heteroatom |
|---|---|---|---|
| polymer | ratio | flow rate | gas source |
| Ethylene: | R = 1 | 10 sccm ethylene | 10 sccm ammonia |
| Nitrogen | | (99.999%) | (99.999%) |
| Ethylene: | R = 4 | 5 sccm ethylene | 20 sccm carbon dioxide |
| Oxygen | | (99.999%) | (99.999%) |

Rods were incubated overnight (20 h total) in the different solutions. Rods were then rinsed with 1 mL f sterile superQ water and left to dry a few hours under the biological hood.

### Example 6a: Plaque Assay

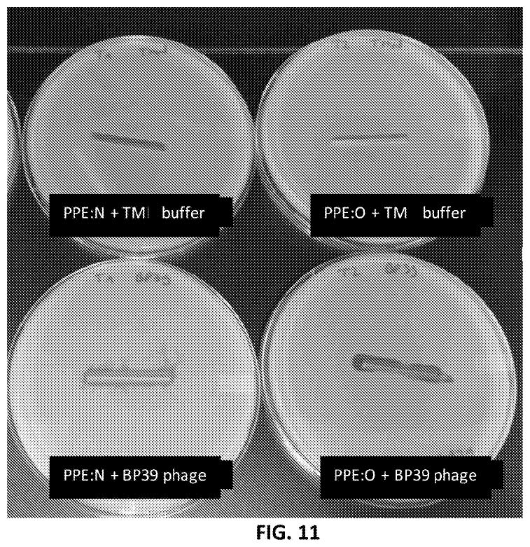

We performed plaque assays to assess the capture of host cells by phages, and assess if the phage particles retained their active conformation. The rods were directly put into contact with a bacterial (SaA29) top agar to assess direct lysis. The plaque assay clearly shows that only the titanium rods treated with either oxygen or nitrogen plasma polymerization and BP39 bacteriophages were able to lyse the bacterial lawn, as opposed to the plasma polymer and TMN buffer treated counterparts (FIG. 11). Covalently immobilized bacteriophages thus retained their infectivity with both types of treatment.

### Example 6b: Liquid Proliferation Assay

This method describes the steps necessary to quantify the amount of Staphylococcus aureus that proliferates onto the test coupons following the attachment assay. The test coupons are incubated with an initial inoculum concentration described in the attachment assay. After the 1 hr period, the coupons are washed and allowed to proliferate and used in subsequent steps to determine CFU/pin and in the proliferation assays. An initial inoculum is used for attachment. Pins rock and incubate for an hour. Then, the pins are gently washed and allowed to proliferate for an additional 18-24 hr in soft agar as a simulation of soft tissue and in a standard liquid assay for an additional 18-24 hr after which time, the CFU/pin is determined

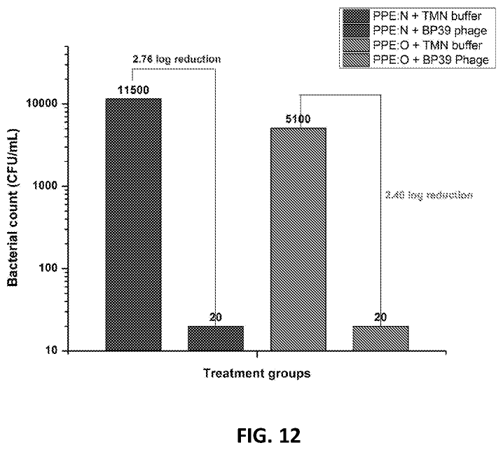

Both PPE:N and PPE:O+phage treatments seem to be very effective with 2.76 and 2.40 bacterial log reduction when compared to the TMN treatment (FIG. 12).

### Example 6c: Soft Agar Proliferation Assay

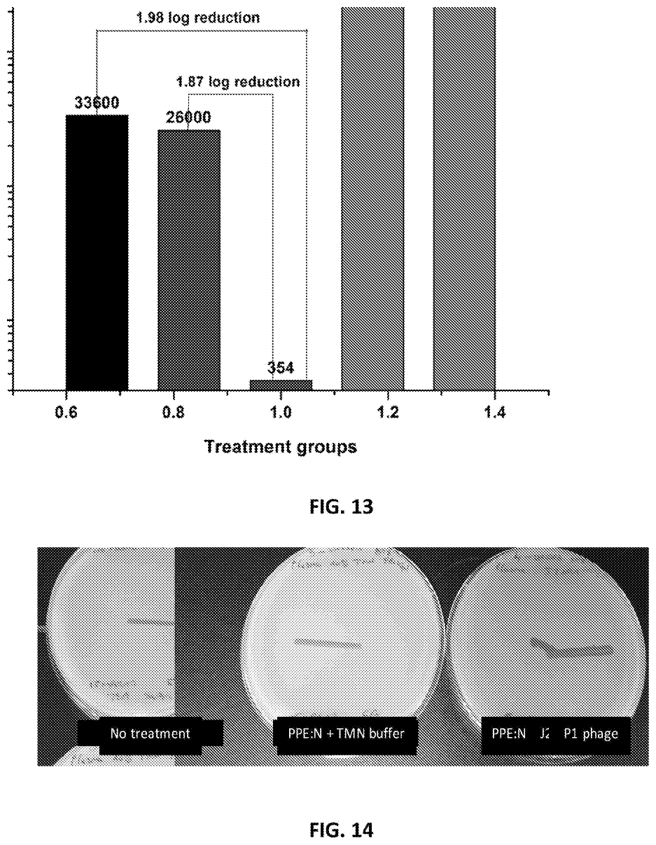

This method describes the steps necessary to quantify the amount of Staphylococcus aureus that proliferates onto the test coupons following the attachment assay. The test coupons are incubated with an initial inoculum concentration described in the attachment assay. After the 1 hour period, the coupons are washed and allowed to proliferate and used in subsequent steps to determine CFU/pin and in the proliferation assays. An initial inoculum is used for attachment. Pins rock and incubate for an hour. Then, the pins are gently washed and allowed to proliferate for an additional 18-24 hr in soft agar as a simulation of soft tissue and in a standard liquid assay for an additional 18-24 hours after which time, the CFU/pin is determined PPE:N+phage showed a 1.98 and 1.87 log reduction when compared to the non-treatment group and the PPE:N+TMN buffer group (FIG. 13).

From the previous set of experiments, it appears that the treatments allow for covalent bonding of bacteriophages or allows for the phage to retain their infectivity.

### Example 6d: X-Ray Photoelectron Spectroscopic Characterization of Bacteriophage-Titanium Rod Complexes

XPS analyses were performed in a Thermo Scientific K-Alpha™ instrument using monochromatic Al Ka radiation, 22-24 h after deposition. Survey spectra were acquired at a pass energy of 160 eV and corrections were done by referencing all peaks with respect to the carbon (C1 s) peak at a binding energy of 285.0 eV. The atomic concentrations, calculated using 2.3.16 PR 1.6 Avantage software, are used to evaluate surface composition. The relative sensitivity factor (RSF) values for carbon, nitrogen, and oxygen are 1, 1.8, and 2.93, respectively. The high resolution XPS peak analysis was done by first applying a Shirley background. The C1 s spectra were fitted with four component peaks (C1-C4) using full-width at half-maximum (FWHM) of 1.2 eV.

Phages were coupled to Ti rods using PPE:N deposition. Chemical alterations of the sample surfaces, leading to the formation of covalent bond between the bacteriophages and the Ti rods, were studied by XPS. An elemental survey of the samples shows the presence of carbon (69.35%), oxygen (15.93%), and nitrogen (14.09%), contributed by the presence of phages. The undetectable atomic percentages of titanium indicates that Ti rods were densely covered by phages. Thus, we expect relatively similar carbon, nitrogen, and oxygen percentages for phage—NPs hybrids when compared with phages.

In addition to measuring the atomic concentration by XPS, high resolution XPS was also performed to determine the chemical bonding states. The curves of high resolution carbon (C1s) peaks were fitted with four sub-peak components (C1-C4) for samples with different R values by constraining the peak position components. Samples chosen were a Ti rod and two Ti rods coated with nitrogen-rich plasma polymer that were dipped in solution, one containing bacteriophages (Ti:PPE:N+Bac) and the other without bacteriophages (Ti-PPE:N). A Si wafer and a droplet of bacteriophage solution on a Si wafer (Si+Bac) were also analysed. All samples that had been in contact with liquids had to be thoroughly dried before analysis.

Samples were analysed on a Thermo Scientific™ K-Alpha™+XPS. Survey spectra were recorded at a resolution of 1 eV and spectra of C1s, N1s and O1s peaks were recorded at a resolution of 0.1 eV.

Data analysis was performed using the Avantage (TM) software (version 5.956). For general composition analysis of each sample, the automatic identification function was used. Results from different points on each sample were then averaged. The error bars represent one standard deviation. For peak fitting, each peak was first charge corrected to have the C1s peak at 285 eV and a Shirley background was applied. Whenever possible for samples Ti-PPE:N and Ti-PPE:N+Bac, the FWHM and component positions were fixed at the values reported in the article by Tawil et al.[1] However, it was not possible to obtain a good fit for Si+Bac using these parameters. Therefore, no constraints were applied to both component position and FWHM for the peak analysis of Si+Bac.

Table 3 presents the binding energies, FWHM and percentage areas of the fitted components for carbon, nitrogen and oxygen on samples Ti-PPE:N, Ti-PPE:N+Bac and Si+Bac.

TABLE 3
Binding energies, FWHM and percentage areas of the fitted components for carbon,
nitrogen and oxygen on samples Ti-PPE:N, Ti-PPE:N + Bac and Si + Bac.

| Peak | | FWHM | Si + Bac | Ti-PPE:N | Ti-PPE:N + Bac | Suggested |
|---|---|---|---|---|---|---|
| component | BE (eV) | (eV) | At % | At % | At % | attribution |
| C1 | 283.00 | 1.54 | | 5.45 | 4.27 | NH3/Ti |
| C2 | 284.00 | | | 5.27 | 10.34 | C2H4/Ti |
| C3 | 284.99 | | 29.97 | 55.44 | 47.16 | Aliphatic carbon |
| C4 | 285.91 | | | 20.57 | 20.66 | C—N; C—S |
| C5 | 286.98 | | 54.63 | 12.06 | 12.93 | C—OH |
| C6 | 288.14 | | 15.4 | 1.21 | 4.65 | O═C—N; O—C—C; COOH |
| N1 | 398.00 | 1.64 | 7.545 | 13.91 | 12.42 | NH3/Ti; NH3/Si |
| N2 | 399.00 | | 63.42 | 50.18 | 42.77 | amine |
| N3 | 400.00 | | 29.13 | 35.92 | 43.05 | amide |
| N4 | 401.00 | | | 0 | 1.76 | Protonated/Oxidized |
| | | | | | | nitrogen |
| O1 | 530.45 | 1.73 | | 20.5 | 20.90 | COO— |
| O2 | 531.69 | | 39.48 | 52.81 | 51.26 | carbonyl |
| O3 | 532.92 | | 60.52 | 26.69 | 27.84 | carboxyl |

The C1 and C2 components are assigned to contribution of Ti on ammonia and ethylene bonds while C3 is representative of aliphatic carbons, C4 includes amines C—N, C═N, and nitriles, C5 includes nitriles, hydroxyl and ether groups (C—O—C═O). Finally, the C6 component is assigned to contributions from carbonyls, namely C═O and N—C═O, as well as contributions from N—C—O. Considering the complexity of plasma polymer films, these assignments should be considered suggestions and other functional groups could be responsible for the observed chemical shifts.

### Example 7: Suitability of the Plasma Method for the Immobilization of Morphologically Dissimilar Bacteriophages

Each phage particle is constituted of genomic material enclosed in a protein or lipoprotein coat, called a capsid. Phages are subdivided in multiple genera, based on their morphology, nucleic acid homology and serology, and two groups, based on their life cycles, replication, and propagation. Lytic bacteriophages are virulent phages that specifically recognize and infect their host bacterium, use the bacterial machinery to replicate their genomes, produce and assemble their structural components, and ultimately lyse and kill the bacterium to release the newly assembled virions. Currently, phages are grouped in nineteen families. Tailed phages, of the order Caudovirales, account for approximately 95% of present-day isolated bacteriophages. The virions have an icosahedral head.

Podoviridae, such as BP39, are a type of phages with short non-contractile tail, while others, such as J21-P1 are myoviridae, with longer contractile tails. Systematic investigations of the relationship between the immobilization conditions and the resulting orientation of these adsorbed phages and their bioreactivity, is of importance as a better understanding of the immobilization process for different types of bacteriophagtes will improve the potential to design better coated implant. We investigated if the same PPE:N method can be employed for the immobilization of both podoviridae and myoviridae on the surface of titanium implants.

### Example 7a: Plaque Assay

The plaque assay clearly shows that only the titanium rods treated with nitrogen plasma polymerization (Treatment 1) and J21-P1 bacteriophages were able to lyse the bacterial lawn, as opposed to the plasma polymer and TMN buffer treated counterparts. As with BP39, J21-P1 covalently immobilized bacteriophages thus retained their infectivity (FIG. 14).

### Example 7b: Atomic Force Microscopy

Peak force tapping mode AFM measurements were performed using Asylum MPF3D with Molecular Force Probe 3D controller ACTA (AppNano) at k: 37 N/m and fₒ: 300 kHz. Untreated Ti rods show smooth surfaces with some granules in agreement with previously published reports on machined Ti-rods.

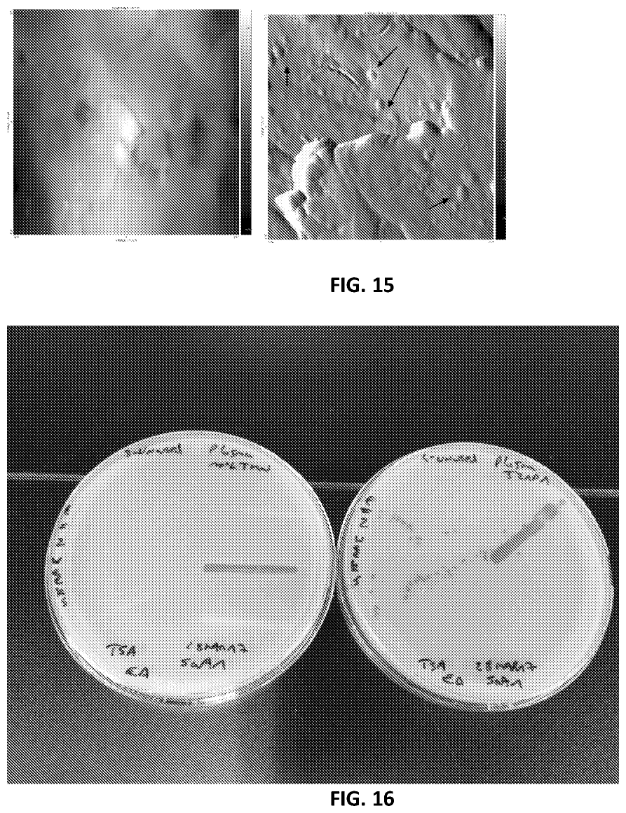

Multiple plasma treated samples have qualitative similar surface topographies which, however, are distinctly different from the untreated Ti rods and Ti-PPE:N+Phage treated rods. The surfaces have a relatively smooth appearance, with clearly visible grains and grain boundaries. While the grains have smooth surfaces, they show a corrugated topography on the submicron level. AFM revealed the presence of compact linear bodies are finely distributed, covering the substrate surface almost completely. J21P1 bacteriophages can clearly be seen on the AFM images of Ti-PPE:N+Bac samples, with dimensions corresponding to J21P1 phages (FIG. 15).

### Example 8: Stability of Coatings

This assay was performed to establish the activity of the immobilized phages by treatment 1 over time. Bacterial SaA1 inoculum will be titrated (10\hat{ }4 asked in the SOPs). For titration in proliferation assays, 150 uL of appropriated dilutions are plated per Petri dish (one dilution/petri). Washed and dried Ti rods were kept in a glass vial at 4° C. until activity was assessed.

### Example 8a: Plaque Assay for 2-Week Stability Time Point

Week two results echoed the results for the previous week and we concluded that the immobilized phages remained active when compared to samples that were not treated with plasma (no activity) (FIG. 16).

### Example 8b: Solid Proliferation Assay for 2-Week Stability Time Point

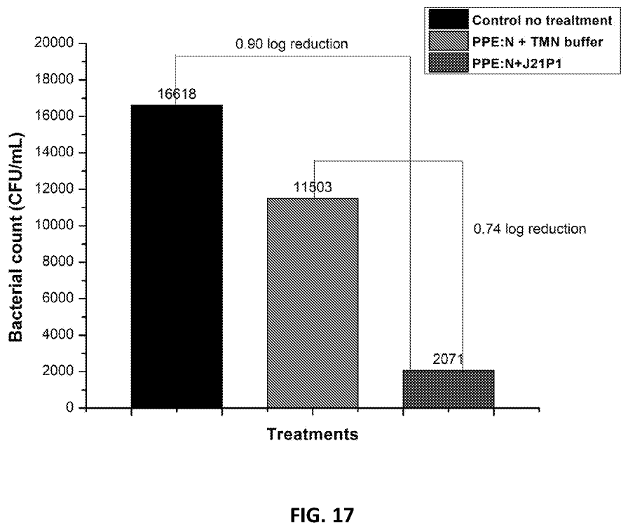

Solid proliferation assay (as described in previous sections) were performed to establish the activity of immobilized phages following plasma treatment 1 at 2 weeks post treatment. Results show maintained activity with 0.74 to 0.9 log reduction subsequent to plasma treatment and phage immobilization (FIG. 17).

### Example 9: Immobilization of Encapsulated Phages Following Plasma Treatment

Plasma Treatment 1: (10 sccm ammoniac, 10 sccm ethylene, P=80 Pa, 20 W, 10 min, ca 100 nm deposit) was applied to Titanium k-wires as described previously. Following plasma treatment, rods were treated with (1) a block copolymer (PEAU) by dip coating (Liquid patch formulation); (2) a formulation containing microencapsulated bacteriophages (Formulation 9) in liquid buffer, (3) Microencapsulated bacteriophages with an added concentration of polyvinyl alcohol (PVA) which enables the formation of a thin film when sprayed on the implant (Formulation 10); (4) and microencapsulated bacteriophages in a formulation containing pluronics, which has the properties of forming a gel when sprayed on the implant (Formulation 11). All these formulations contain a cocktail of 15 different lytic phages targeting S. aureus, P. aeruginosa, and K. pneumoniae (Table 4).

Rods were cleaning and sterilized in the following fashion: Rods were sonicated for 15 min in 3 mL isopropanol in 15 mL Falcon tubes in a sonication bath. Furthermore, rods were sonicated for 15 min in 3 mL sterile superQ water in new 15 mL Falcon tubes. Rods were rinsed 2× in sterile superQ water in new 15 mL and dried under the biological hood. All rods will be autoclaved for 15 min at 121° C. (grav15) individually in 2 mL glass vials. Plasma is generated with a stainless-steel electrode. Pre-deposition is made with treatment 1 (no samples). Rods, petri dishes, support, sterile tweezers are inserted in the box via the chamber. Rods are placed vertically on a in-house made support so they don't touch each other. Support is positioned on the electrode. Treatment 1 is applied.

After plasma treatment, rods are inserted in the corresponding formulation (Liquid patch, or Formulation 9) (3 mL of solution in a 15 mL falcon tub) or sprayed with 15 mL of Formulation 10 or Formulation 11 under the biological hood. Rods are withdrawn gently from the formulations and placed back in small sterile GC vial (the treated zone does not touch the glass). Rods were dried overnight under the laminar flow hood. Release of phages was monitored in EMEM media (supplemented with 10% FBS) at 37° C. and was assessed for 4 weeks by titration on S. aureus, P. aeruginosa, and K. Pneumoniae.

The dip coating strategy, as well as the spray-on microencapsulated strategy all showed controlled release of bacteriophages for over 672 hours (over 28 days), a result not shown for directly immobilized phages on the titanium substrates (Table 4).

TABLE 4
Release of bacteriophages from immobilized formulations.

| | Time(h) | | | | | | | | | |
|---|---|---|---|---|---|---|---|---|---|---|
| Description | 0 | 0.5 | 1 | 2 | 6 | 24 | 48 | 120 | 144 | 168 |

K. pneumoniae

| | | | | | | | | | | |
|---|---|---|---|---|---|---|---|---|---|---|
| No plasma + No | 1.9E+04 | 5.1E+03 | 0.0E+00 | 0.0E+00 | 0.0E+00 | 0.0E+00 | 0.0E+00 | 0.0E+00 | 0.0E+00 | 0.0E+00 |
| phages | | | | | | | | | | |
| Plasma + No | 0.0E+00 | 0.0E+00 | 0.0E+00 | 0.0E+00 | 0.0E+00 | 0.0E+00 | 0.0E+00 | 0.0E+00 | 0.0E+00 | 0.0E+00 |
| phages | | | | | | | | | | |
| Plasma + | 2.1E+06 | 3.7E+05 | 3.8E+03 | 3.9E+03 | 1.1E+04 | 1.5E+03 | 1.0E+02 | 1.1E+03 | — | — |
| Formulation 9 | | | | | | | | | | |
| Plasma + | 5.2E+06 | 1.5E+05 | 5.3E+03 | 2.0E+04 | 3.9E+03 | 1.0E+03 | 2.0E+03 | 1.0E+02 | — | — |
| Formulation 10 | | | | | | | | | | |
| Plasma + | 2.8E+07 | 5.6E+04 | 1.4E+04 | 1.3E+03 | 1.2E+03 | 1.0E+02 | 1.0E+02 | 1.0E+02 | 1.0E+02 | — |
| Formulation 11 | | | | | | | | | | |
| Plasma + | 2.4E+05 | 2.7E+05 | 1.5E+05 | 1.7E+05 | 4.9E+05 | 6.2E+04 | 1.8E+05 | 1.0E+05 | 2.1E+04 | 3.1E+04 |
| Liquid Patch | | | | | | | | | | |

S. aureus

| | | | | | | | | | | |
|---|---|---|---|---|---|---|---|---|---|---|
| Plasma + No | 0.0E+00 | 0.0E+00 | 0.0E+00 | 0.0E+00 | 0.0E+00 | 0.0E+00 | 0.0E+00 | 0.0E+00 | 0.0E+00 | 0.0E+00 |
| phages | | | | | | | | | | |
| Plasma + | 3.6E+04 | 1.6E+05 | 1.7E+04 | 4.0E+04 | 6.2E+04 | 2.9E+04 | 2.2E+04 | 2.8E+03 | 2.2E+03 | 2.1E+03 |
| Liquid Patch | | | | | | | | | | |
| Plasma + | 0.0E+00 | 1.3E+04 | 3.2E+03 | 5.9E+03 | 7.6E+03 | 4.4E+03 | 3.8E+03 | 1.3E+03 | 1.0E+02 | 1.0E+02 |
| Liquid Patch | | | | | | | | | | |

P. aeruginosa

| | | | | | | | | | | |
|---|---|---|---|---|---|---|---|---|---|---|
| No plasma + | 2.3E+05 | 2.1E+04 | 0.0E+00 | 0.0E+00 | 0.0E+00 | 0.0E+00 | 0.0E+00 | 0.0E+00 | 0.0E+00 | 0.0E+00 |
| No phages | | | | | | | | | | |
| Plasma + No | 1.7E+03 | 0.0E+00 | 0.0E+00 | 0.0E+00 | 0.0E+00 | 0.0E+00 | 0.0E+00 | 0.0E+00 | 0.0E+00 | 0.0E+00 |
| phages | | | | | | | | | | |
| Plasma + | 1.5E+07 | 3.1E+05 | 1.1E+04 | 5.8E+03 | 6.1E+03 | 1.8E+03 | 1.1E+03 | 1.0E+02 | 1.0E+02 | 1.0E+02 |
| Formulation 9 | | | | | | | | | | |
| Plasma + | 1.3E+07 | 4.2E+05 | 3.8E+04 | 4.8E+04 | 2.7E+04 | 3.1E+03 | 5.2E+03 | 1.2E+03 | 1.0E+02 | 1.0E+02 |
| Formulation 10 | | | | | | | | | | |
| Plasma + | 2.8E+07 | 1.2E+05 | 3.8E+03 | 4.4E+03 | 3.5E+03 | 1.0E+02 | 1.0E+02 | 1.0E+02 | — | — |
| Formulation 11 | | | | | | | | | | |
| Plasma + | 2.1E+05 | 2.0E+05 | 1.2E+05 | 1.9E+05 | 4.6E+05 | 1.2E+05 | 4.7E+04 | 4.6E+04 | 3.7E+03 | 2.7E+04 |
| Liquid Patch | | | | | | | | | | |

### Example 10: Preventing Biofilm Infections

SaA3 Staphylococcus aureus strain was used in the biofilm assays. A bacteriophage cocktail targeting S. aureus, P. aeruginosa, and K. pneumoniae was used. Formulations in which the phages are encapsulated in microspheres are also used for the tests, which contain the same cocktail of phages. These are F11 (gel-forming spray). Titanium and stainless steel k-wires were cut using Exakt Cutting tool in order to get disks that could be used in 96-well microplates. The disks are sonicated for 15 minutes in water followed by ethanol and water again, to The discs are then autoclaved. The discs underwent plasma polymerization (Treatment R=2 (10 sccm ammoniac, 20 sccm ethylene, P=80 Pa, 20 W, 10 min, ca 100 nm deposit) or R=8 (10 sccm ammoniac, 80 sccm ethylene, P=80 Pa, 20 W, 10 min, ca 100 nm deposit). Following treatment, the discs were incubated with either formulation 11, or the phage cocktail for an hour. The disks were then washed 3 times, dried under the laminar flow hood and transferred to a new 96-well plate. The culture medium of the biofilm inserted into the wells is obtained by adding 1% glucose (1 g/100 ml) to the TSB medium. The discs are placed in the bottom of the wells of the microplate on their flat surface. For the negative control 100 μl of the culture medium is placed in the wells. Positive control is 90 μl of the culture medium and 10 μl of bacterial suspension solution. The plates are incubated at 37° C. for 24 hours.

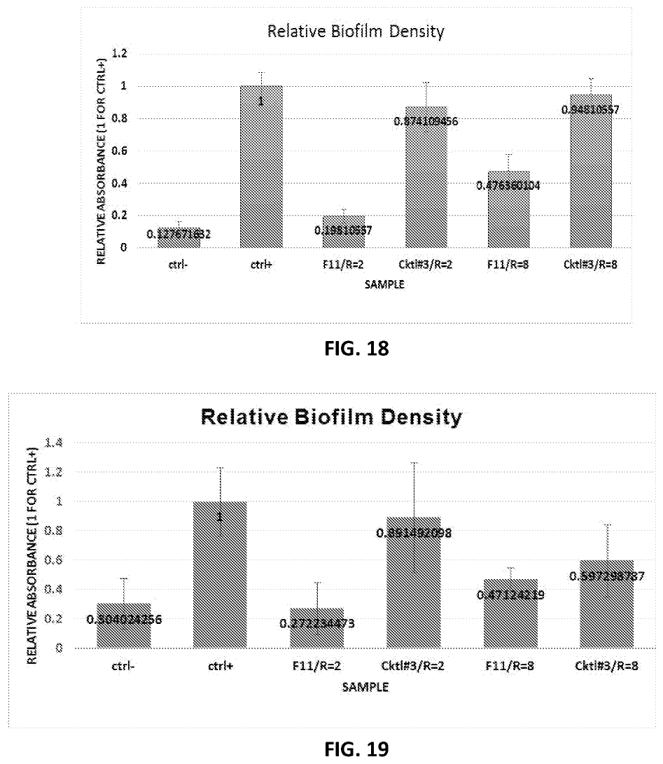

The contents of the wells are aspirated using a vacuum pump and pastoral pipettes, taking care not to disturb the potentially formed biofilm by avoiding touching the surface of the discs. The wells are filled with 100 μL PBS at room temperature, and aspirated again. This step is repeated 3 times Biofilms are fixed in an oven at 60° C. The wells are filled with 100 μl of methanol, and incubated for 20 minutes, before the content is aspirated. The plates are allowed to dry overnight. The potentially formed biofilm is stained with 2% crystal violet and then discs are treated with 95% methanol and incubated for 30 minutes. Optical density is read at 570 nm (FIGS. 17 and 18).

Pre-treatment of phages and their ability to resolve biofilm formation were studied. Results demonstrated that pre-treatment with formulation F11 following plasma polymerization provided an effective way of significantly reducing or preventing all together biofilm formation on implants. Direct immobilization of phages was ineffective for preventing biofilm formation.

Although the present invention has been described hereinabove by way of preferred embodiments thereof, it can be modified, without departing from the spirit and nature of the subject invention as defined in the appended claims. Also, although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole.

1. A medical device, the medical device comprising:
a substrate defining a surface;
a plasma polymer layer bound to and coating the surface; and
a bactericide layer bound to the plasma polymer layer, the plasma polymer layer being between the substrate and the bactericide layer.

2. The medical device as defined in claim 1, wherein the bactericide layer includes bioactive bacteriophages.

3. The medical device as defined in claim 1, wherein the bactericide layer includes bacteriophage related products selected from the group consisting of endolysins, lysostaphins, phage proteins, phage enzymatic formulations, and combinations thereof.

4. The medical device as defined in claim 1, wherein the bactericide layer further includes a bioactive agent selected from the group consisting of antibiotics, a cell adhesion promoting agents, an antithrombic factors, antiseptics, anti-infectives, antibiotics, pain relievers, antibacterials, antiprotozoal agents, antiviral agents, analgesics, anti-inflammatory agents, contraceptives, CNS active drugs, hormones, enzymes, hemostatics, and vaccines.

5. (canceled)

6. (canceled)

7. The medical device as defined in claim 5 wherein the substrate is essentially made of titanium.

8. The medical device as defined in claim 5, wherein the substrate includes at least one of a polymer, iron, copper, zinc, lead, aluminum, titanium, gold, platinum, silver, cobalt, chromium, vanadium, tantalum, nickel, magnesium, manganese, cobalt chrome, nickel titanium, titanium vanadium aluminum, and stainless steel.

9. The medical device as defined in any one of claims 1 to 8, wherein the plasma polymer layer is between 10 and 1000 nm thick, optionally between 100 and 500 nm thick.

10. (canceled)

11. The medical device as defined in claim, wherein the bactericide layer includes bioactive bacteriophages covalently bound to the plasma polymer layer, optionally a combination of both podoviridae and myoviridae in the same bactericide layer.

12. The medical device as defined in claim 1, wherein the bactericide layer includes bacteriophage related products covalently bound to the plasma polymer layer.

13. The medical device as defined in claim 1, wherein the bactericide layer includes a coating material covalently or electrostatically, or both covalently and electrostatically bound to the plasma polymer layer.

14. (canceled)

15. The medical device as defined in claim 1, wherein the bactericide layer includes bioactive bacteriophages dispersed in a coating material, the coating material being bound to the plasma polymer, optionally wherein the coating material is a polymer defining an exposed surface, the bactericide layer defining microchannels extending in the coating material from the exposed surface, further optionally wherein the microchannels have a diameter of from about 5 nm to about 5 μm.

16. The medical device as defined in claim 1, wherein the bactericide layer includes bacteriophage related products dispersed in a coating material, the coating material being bound to the plasma polymer, optionally wherein the coating material is a polymer defining an exposed surface, the bactericide layer defining microchannels extending in the coating material from the exposed surface, further optionally wherein the microchannels have a diameter of from about 5 nm to about 5 μm.

17. (canceled)

18. (canceled)

19. The medical device as defined in claim 15, further comprising salt crystals embedded in the coating material.

20. The medical device as defined in claim 19, wherein the bacteriophages are adsorbed on the salt crystals.

21. The medical device as defined in claim 19, wherein the salt crystals include at least one of calcium salt crystals, magnesium salt crystals, strontium salt crystals, and barium salt crystals.

22. The medical device as defined in claim 19, wherein the salt crystals are between about 5 nm and about 5 μm in size.

23. The medical device as defined in claim 15, wherein the coating material is a block co-polymer.

24. The medical device as defined in claim 1, wherein the bactericide layer includes bacteriophage-containing biodegradable microcapsules bound to the plasma polymer.

25. The medical device as defined in claim 1, wherein the bactericide layer includes bacteriophage-containing biodegradable microcapsules embedded in a coating material, the coating material being bound to the plasma polymer, the bactericide layer optionally including bacteriophages dispersed thereinto outside of the microcapsules, optionally wherein the biodegradable microcapsules are made of a co-polymer.

26. (canceled)

27. (canceled)

28. (canceled)

29. The medical device as defined in claim 1, wherein the medical device is selected from the group consisting of an orthopaedic implant, a stent, a catheter, and a defibrillator.

30.-72. (canceled)

73. A medical device, the medical device comprising:
a substrate defining a surface;
a bactericide layer sprayed on the surface, the bactericide layer containing microencapsulated bacteriophages.
